SEQUENCE LISTING (22 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 14 aa, Homo sapiens
SKRNIQFSCKNIYR

SEQ ID NO: 3 - protein, 14 aa, Homo sapiens
EKVQTLEAWVIHGG

SEQ ID NO: 4 - protein, 122 aa, Artificial Sequence [sequence omitted]

SEQ ID NO: 5 - protein, 107 aa, Artificial Sequence [sequence omitted]

SEQ ID NO: 6 - protein, 5 aa, Artificial Sequence
  daratumumab HCDR1
SFAMS

SEQ ID NO: 7 - protein, 17 aa, Artificial Sequence
  Daratumumab HCDR2
AISGSGGGTYYADSVKG

SEQ ID NO: 8 - protein, 13 aa, Artificial Sequence
  Daratumumab HCDR3
DKILWFGEPVFDY

SEQ ID NO: 9 - protein, 11 aa, Artificial Sequence
  Daratumumab LCDR1
RASQSVSSYLA

SEQ ID NO: 10 - protein, 7 aa, Artificial Sequence
  Daratumumab LCDR2
DASNRAT

SEQ ID NO: 11 - protein, 10 aa, Artificial Sequence
  Daratumumab LCDR3
QQRSNWPPTF

SEQ ID NO: 12 - protein, 452 aa, Artificial Sequence
  Daratumumab heavy chain
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Artificial Sequence
  Daratumumab light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 122 aa, Artificial Sequence
  mAb 003 VH
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 15 - protein, 107 aa, Artificial Sequence
  mAb 003 VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 16 - protein, 122 aa, Artificial Sequence
  mAb024 VH
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, Artificial Sequence
  mAb 024 VL
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 18 - protein, 120 aa, Artificial Sequence
  MOR202 VH
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 19 - protein, 109 aa, Artificial Sequence
  MRO202 VL
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 20 - protein, 120 aa, Artificial Sequence [sequence omitted]

SEQ ID NO: 21 - protein, 107 aa, Artificial Sequence [sequence omitted]

SEQ ID NO: 22 - protein, 509 aa, Artificial Sequence
  rHuPH20
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

(19) United States
(12) Patent Application Publication
(10) Pub. No.: US 2020/0121588 A1
(43) Pub. Date: Apr. 23, 2020

(54) Method of Providing Subcutaneous Administration of Anti-CD38 Antibodies

(71) Applicant: Janssen Biotech, Inc., Horsham, PA (US)

(72) Inventors: Kim Anne Campbell, Downingtown, PA (US); Ivo Nnane, East Fallowfield, PA (US); Donald Raible, Berwyn, PA (US); Cathye Shu, Landsdale, PA (US); Zhenhua Xu, Berwyn, PA (US)

(21) Appl. No.: 16/656,569

(22) Filed: Oct. 17, 2019

Related U.S. Application Data
(60) Provisional application No. 62/747,107, filed on Oct. 17, 2018.

(51) Int. Cl. A61K 9/00; A61K 31/573; A61K 38/47; C07K 16/28; A61P 37/06

(52) U.S. Cl. CPC A61K 9/0019; A61K 31/573; C12Y 302/01035; C07K 16/2896; A61P 37/06; A61K 38/47

(57) ABSTRACT

Provided are methods of subcutaneous administration of anti-CD38 monoclonal antibodies, such as daratumumab. Also provided are methods for the treatment of autoimmune diseases with subcutaneously administered anti-CD38 monoclonal antibodies. A corticosteroid is optionally administered prior to subcutaneous administration of the anti-CD38 antibody to prevent and/or reduce systemic injection related side effects.

### RELATED APPLICATION

This application claims the benefit of U.S. Provisional Application No. 62/747,107, filed on Oct. 17, 2018. The entire teachings of the above application are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:

a) File name: 01482025002 SequenceListing.txt; created 10/16/2019, 26 KB in size.

The invention relates to methods of providing subcutaneous administration of anti-CD38 antibodies and methods of providing treatment of an autoimmune disease by subcutaneous administration of anti-CD38 antibodies.

### BACKGROUND

CD38 is a multifunctional protein having functions in receptor-mediated adhesion and signaling as well as mediating calcium mobilization via its ecto-enzymatic activity, catalyzing formation of cyclic ADP-ribose (cADPR) and ADP-ribose (ADPR). CD38 mediates cytokine secretion and activation, and proliferation of lymphocytes (Funaro et al., J Immunolog 145:2390-96, 1990; Terhorst et al., Cell 771-80, 1981; Guse et al., Nature 398:70-73, 1999). CD38, via its nicotinamide adenine dinucleotide (NAD) glycohydrolase activity, also regulates extracellular NAD⁺ levels, which have been implicated in modulating the regulatory T-cell compartment (Adriouch et al., Microbes Infect. 14: 1284-92, 2012; Chiarugi et al., Nature Reviews 12: 741-52, 2012). In addition to signaling via Ca²⁺, CD38 signaling occurs via cross-talk with antigen-receptor complexes on T- and B-cells or other types of receptor complexes, e.g., MEW molecules, involving CD38 in several cellular responses, but also in switching and secretion of IgG1.

CD38 is a type II transmembrane glycoprotein expressed on hemopoietic cells such as medullary thymocytes, activated T- and B-cells, resting natural killer (NK) cells and monocytes, lymph node germinal center lymphoblasts, plasma B cells, intrafollicular cells and dendritic cells. A portion of normal bone marrow cells, particular precursor cells as well as umbilical cord cells are CD38-positive. In addition to lymphoid precursor cells, CD38 is expressed on erythrocytes and on platelets, and expression is also found in some solid tissues such as gut, brain, prostate, bone, and pancreas. Mature resting T- and B-cells express limited to no surface CD38.

CD38 is also expressed in a variety of malignant hematological diseases, including multiple myeloma, leukemias and lymphomas, such as B-cell chronic lymphocytic leukemia, T- and B-cell acute lymphocytic leukemia, Waldenstrom macroglobulinemia, primary systemic amyloidosis, mantle-cell lymphoma, pro-lymphocytic/myelocytic leukemia, acute myeloid leukemia, chronic myeloid leukemia, follicular lymphoma, Burkitt's lymphoma, large granular lymphocytic (LGL) leukemia, NK-cell leukemia and plasma-cell leukemia. Expression of CD38 has been described on epithelial/endothelial cells of different origin, including glandular epithelium in prostate, islet cells in pancreas, ductal epithelium in glands, including parotid gland, bronchial epithelial cells, cells in testis and ovary and tumor epithelium in colorectal adenocarcinoma. Other diseases, where CD38 expression may be involved, include, e.g., broncho-epithelial carcinomas of the lung, breast cancer (evolving from malignant proliferation of epithelial lining in ducts and lobules of the breast), pancreatic tumors evolving from the beta-cells (insulinomas), tumors evolving from epithelium in the gut (e.g., adenocarcinoma and squamous cell carcinoma), carcinoma in the prostate gland, and seminomas in testis and ovarian cancers. In the central nervous system, neuroblastomas express CD38.

DARZALEX® (daratumumab) is a human immunoglobulin G1 kappa (IgG1K) monoclonal antibody (mAb) that binds to CD38. It is a targeted immunotherapy that depletes tumor cells that express high levels of CD38, by a variety of mechanisms, including complement dependent cytotoxicity (CDC), antibody dependent cell mediated cytotoxicity (ADCC), and antibody dependent cellular phagocytosis (ADCP) and approved as a monotherapy or as a combination therapy for treating patients with multiple myeloma according to the prescribing information in the US and in other countries.

The safety profile of daratumumab has thus been well characterized in MM patients when administered intravenously. Studies on the safety of subcutaneously administered daratumumab to multiple myeloma patients have also been conducted. However, the expression of CD38 is higher in oncology patients, such as patients with MM, as compared to the CD38 levels observed in healthy subjects and patients with other diseases. Due to the increased level of CD38 expression, relatively high doses of daratumumab were subcutaneously administered to MM patients, necessitating combining daratumumab with recombinant human hyaluronidase to facilitate administration. Thus, the pharmacokinetics (PK) and pharmacodynamics (PD) of daratumumab in patients with MM is likely to be very different from those in a non-oncology population due to the higher CD38⁺ cell burden in oncology patients.

In rheumatoid arthritis (RA), CD38 and plasma cell-related genes are highly expressed in the synovial tissue of RA patients at all the stages of the disease (Owczarczyk et al., Sci Transl. Med. 3(101): 101ra92, 2011; Chang et al. Clin. Exp. Immunol. 176(2): 222-31, 2014). Plasma cells have a pathogenic role as the main producers of autoantibodies, such as anti-citrullinated peptide antibodies (ACPAs) and rheumatoid factor (RF) (autoantibodies against the fragment crystallizable (Fc) portion of IgG molecules) and antibodies to other structural proteins in RA (Aletaha et al. Arthritis Res. Ther. 17: 229, 2015; Gonzalez et al. J. Rheumatol. 35: 1009-14; 2008; van Gaalen et al. Arthritis Rheum. 50: 2113-21, 2004; Barra et al. Rheumatology, 50: 311-16, 2011; Nell-Duxneuner et al. Ann. Rheum. Dis., 69: 169-74, 2010). Thus, CD38 may be targeted to selectively deplete and modify plasma cells to affect pathogenesis of RA and other autoimmune diseases. However, the types of cells expressing CD38 (e.g., plasmablasts/plasma cells (PB_PC), NK cells, B-cells, T-cells, etc.), and levels of CD38 cell surface expression in healthy adults are comparable with those in adults with RA (Honda et al. Annu. Rev. Immunol. 30: 759-95, 2012; Chang et al. Clin. Exp. Immunol. 176(2): 222-31, 2014). Accordingly, the PK and PD of daratumumab in oncology patients are unlikely to be representative of that in patients with RA and other autoimmune diseases, such as systemic lupus erythematosus (SLE).

Therefore, there is a need to determine the dosage range of an anti-CD38 antibody, such as daratumumab, that can be safely administered, particularly via the subcutaneous route, to a subject diagnosed with or suspected of having an autoimmune disease, such as RA or SLE.

### SUMMARY

The invention relates to subcutaneous administration of an anti-CD38 antibody, such as daratumumab, to a subject, such as a subject diagnosed with or suspected of having an autoimmune disease.

One general aspect of the invention relates to a method of providing subcutaneous administration of an anti-CD38 antibody to a subject in need thereof, the method comprising subcutaneously administering to the subject a pharmaceutical composition comprising the anti-CD38 antibody and a pharmaceutically acceptable carrier, wherein a total dosage of the anti-CD38 antibody administered is about 10 mg to about 2,400 mg per administration.

Another general aspect of the invention relates to a method of providing treatment of an autoimmune disease in a subject in need thereof, the method comprising subcutaneously administering to the subject a pharmaceutical composition comprising an anti-CD38 antibody and a pharmaceutically acceptable carrier, wherein a total dosage of the anti-CD38 antibody administered is about 10 mg to about 2,400 mg per administration.

Yet another general aspect of the invention relates to a method of providing subcutaneous administration of an anti-CD38 antibody to a subject in need thereof, the method comprising subcutaneously administering to the subject a pharmaceutical composition comprising the anti-CD38 antibody and a pharmaceutically acceptable carrier, wherein the anti-CD38 antibody is subcutaneously administered without recombinant human hyaluronidase.

In some embodiments, the anti-CD38 antibody comprises heavy chain complementarity determining region 1 (HCDR1), HCDR2 and HCDR3 having amino acid sequences of SEQ ID NOs: 6, 7 and 8, respectively, and light chain complementarity determining region 1 (LCDR1), LCDR2 and LCDR3 having amino acid sequences of SEQ ID NOs: 9, 10 and 11, respectively.

In some embodiments, the total dosage of the anti-CD38 antibody per administration is administered in a single subcutaneous injection.

In some embodiments, the total dosage of the anti-CD38 antibody per administration is administered in multiple subcutaneous injections, such as 2, 3, 4, or 5 subcutaneous injections.

In some embodiments, a corticosteroid is administered, preferably orally, to the subject prior to the administration of the anti-CD38 antibody, and is optionally re-administered subsequent to the administration of the anti-CD38 antibody.

In some embodiments, the corticosteroid is prednisone.

In some embodiments, the administration of the anti-CD38 antibody results in less than 80% depletion of natural killer (NK) cells or plasma cells four (4) weeks after administration of the anti-CD38 antibody.

In some embodiments, the administration of the anti-CD38 antibody results in a greater than 80% depletion of natural killer (NK) cells or plasma cells for four (4) weeks or less.

In other embodiments, the anti-CD38 antibody is subcutaneously administered without recombinant human hyaluronidase.

In some embodiments, the subject in need thereof has or is suspected of having a disease selected from the group consisting of lupus, systemic lupus erythematosus, Sjogren's Syndrome, arthritis, rheumatoid arthritis, asthma, COPD, pelvic inflammatory disease, Alzheimer's Disease, inflammatory bowel disease, Crohn's disease, ulcerative colitis, Peyronie's Disease, celiac disease, gallbladder disease, Pilonidal disease, peritonitis, psoriasis, psoriatic arthritis, vasculitis, surgical adhesions, stroke, Type I Diabetes, Lyme disease, meningoencephalitis, autoimmune uveitis, multiple sclerosis, Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease, transplant rejection, heart disease including ischaemic diseases such as myocardial infarction as well as atherosclerosis, intravascular coagulation, bone resorption, osteoporosis, osteoarthritis, periodontitis and hypochlorhydia, infertility related to lack of fetal-maternal tolerance, vitiligo, myasthenia gravis or systemic sclerosis.

According to embodiments of the invention, the autoimmune disease is selected from the group consisting of arthritis, rheumatoid arthritis (RA), psoriatic arthritis, ankylosing spondylitis, ulcerative colitis, plaque psoriasis, systemic lupus erythematosus (SLE), lupus nephritis, antineutrophil cytoplasmic antibody (ANCA) associated vasculitis, myasthenia gravis, progressive multiple sclerosis, IgG4 related diseases, Sjogren's syndrome, immune thrombocytopenic purpura, transplant rejection, inflammatory bowel disease and Crohn's disease.

In some embodiments, the autoimmune disease is RA.

In some embodiments, the autoimmune disease is SLE.

Preferably, the subject is a human.

### DRAWINGS

FIGS. 1A-1B depict the effects of daratumumab on CD38⁺ NK cell counts. CD38⁺ NK (CD56⁺) cells were enumerated via flow cytometry on PBMCs isolated from whole blood samples collected longitudinally from subjects treated with placebo or a single administration of daratumumab on Day 0. Total NK cell numbers are shown as the median count of CD38⁺ cells within the CD45⁺CD3⁻CD56⁺ cell population. FIG. 1A The subjects were treated with placebo or a single administration of daratumumab on Day 0 without prednisone. FIG. 1B The subjects were treated with placebo or a single administration of daratumumab on Day 0 combined with 40 mg oral prednisone.

FIGS. 2A-2B depict the effects of daratumumab on the percentage of CD38⁺ NK (CD56⁺) cells. CD38⁺ NK (CD56⁺) cells were enumerated via flow cytometry on PBMCs isolated from whole blood samples collected longitudinally from subjects treated with placebo or a single administration of daratumumab on Day 0. Shown are the percentages of CD38⁺ NK cells remaining after treatment compared to baseline within the parent CD45⁺CD3⁻CD56⁺CD38⁺ cell population. Note that the majority (>95%) of peripheral NK cells are CD38⁺. FIG. 2A The subjects were treated with placebo or a single administration of daratumumab on Day 0 without prednisone. FIG. 2B The subjects were treated with placebo or a single administration of daratumumab on Day 0 combined with 40 mg oral prednisone.

FIGS. 3A-3B depict the effects of daratumumab on CD38⁺ plasmablast/plasma (PB_PC) cell counts. CD38⁺ PB_PC cells were enumerated via flow cytometry on PBMCs isolated from whole blood samples collected longitudinally from subjects treated with placebo or a single administration of daratumumab on Day 0. Total PB_PC cell numbers are shown as the median count of CD38⁺ cells within the CD45⁺CD3⁻CD19+CD20⁻CD27⁺IgD⁻ cell population. FIG. 3A The subjects were treated with placebo or a single administration of daratumumab on Day 0 without prednisone. FIG. 3B The subjects were treated with placebo or a single administration of daratumumab on Day 0 combined with 40 mg oral prednisone.

FIGS. 4A-4B depict the effects of daratumumab on the percentage of CD38⁺ PB_PC cells. CD38⁺ PB_PC cells were enumerated via flow cytometry on PBMCs isolated from whole blood samples collected longitudinally from subjects treated with placebo or a single administration of daratumumab on Day 0. Shown are the percentages of CD38⁺ PB_PC cells remaining after treatment compared to baseline within the parent CD45⁺CD3⁻CD19⁺CD20⁻CD27⁺IgD⁻CD38⁺ cell population. FIG. 4A The subjects were treated with placebo or a single administration of daratumumab on Day 0 without prednisone. FIG. 4B The subjects were treated with placebo or a single administration of daratumumab on Day 0 combined with 40 mg oral prednisone.

FIGS. 5A-5B depict the effects of daratumumab on CD38⁺ lymphocyte cell counts. CD38⁺ lymphocytes were enumerated via flow cytometry on PBMCs isolated from whole blood samples collected longitudinally from subjects treated with placebo or a single subcutaneous administration of daratumumab on Day 0. Total lymphocyte numbers are shown as the median count of CD38⁺ cells within the CD45⁺ cell population. FIG. 5A The subjects were treated with placebo or a single administration of daratumumab on Day 0 without prednisone. FIG. 5B The subjects were treated with placebo or a single administration of daratumumab on Day 0 combined with 40 mg oral prednisone.

FIGS. 6A-6B depict effect of daratumumab on the percentage of CD38⁺ lymphocyte cells. CD38⁺ lymphocytes were enumerated via flow cytometry on PBMCs isolated from whole blood samples collected longitudinally from subjects treated with placebo or a single administration of daratumumab on Day 0. Shown are the percentages of CD38⁺ lymphocyte cells remaining after treatment compared to baseline within the parent CD45⁺CD38⁺ cell population. FIG. 6A The subjects were treated with placebo or a single administration of daratumumab on Day 0 without prednisone. FIG. 6B The subjects were treated with placebo or a single administration of daratumumab on Day 0 combined with 40 mg oral prednisone.

FIGS. 7A-7B depict the effect of daratumumab on CD38⁺ T lymphocyte cell counts. CD38⁺ T lymphocytes were enumerated via flow cytometry on PBMCs isolated from whole blood samples collected longitudinally from subjects treated with placebo or a single administration of daratumumab on Day 0. Total T lymphocyte numbers are shown as the median count of CD38⁺ cells within the CD45⁺CD3⁺ cell population. FIG. 7A The subjects were treated with placebo or a single administration of daratumumab on Day 0 without prednisone. FIG. 7B The subjects were treated with placebo or a single administration of daratumumab on Day 0 combined with 40 mg oral prednisone.

FIGS. 8A-8B depict the effects of daratumumab on the percentage of CD38⁺ T lymphocyte cells. CD38⁺ T lymphocytes were enumerated via flow cytometry on PBMCs isolated from whole blood samples collected longitudinally from subjects treated with placebo or a single administration of daratumumab on Day 0. Shown are the percentages of CD38⁺ T lymphocyte cells remaining after treatment compared to baseline within the parent CD45⁺CD3⁺CD38⁺ cell population. FIG. 8A The subjects were treated with placebo or a single administration of daratumumab on Day 0 without prednisone. FIG. 8B The subjects were treated with placebo or a single administration of daratumumab on Day 0 combined with 40 mg oral prednisone.

FIGS. 9A-9B depict the effects of daratumumab on CD38⁺ B lymphocyte cell counts. CD38⁺ B lymphocytes were enumerated via flow cytometry on PBMCs isolated from whole blood samples collected longitudinally from subjects treated with placebo or a single administration of daratumumab on Day 0. Total B lymphocyte numbers are shown as the median count of CD38⁺ cells within the CD45⁺CD19⁺ cell population. FIG. 9A The subjects were treated with placebo or a single administration of daratumumab on Day 0 without prednisone. FIG. 9B The subjects were treated with placebo or a single administration of daratumumab on Day 0 combined with 40 mg oral prednisone.

FIGS. 10A-10B depict the effects of daratumumab on the percentage of CD38⁺ B lymphocyte cells. CD38⁺ B lymphocytes were enumerated via flow cytometry on PBMCs isolated from whole blood samples collected longitudinally from subjects treated with placebo or a single administration of daratumumab on Day 0. Shown are the percentages of CD38⁺ B lymphocyte cells remaining after treatment compared to baseline within the parent CD45⁺CD19+CD38⁺ cell population. FIG. 10A The subjects were treated with placebo or a single administration of daratumumab on Day 0 without prednisone. FIG. 10B The subjects were treated with placebo or a single administration of daratumumab on Day 0 combined with 40 mg oral prednisone.

FIGS. 11A-11B depict the effects of daratumumab on CD38⁺ monocyte cell counts. CD38⁺ monocyte cells were enumerated via flow cytometry on PBMCs isolated from whole blood samples collected longitudinally from subjects treated with placebo or a single administration of daratumumab on Day 0. Total monocyte cell numbers are shown as the median count of CD38⁺ cells within the CD45⁺CD3⁻CD19⁻CD56⁻CD14+CD16⁻ cell population. FIG. 11A The subjects were treated with placebo or a single administration of daratumumab on Day 0 without prednisone. FIG. 11B The subjects were treated with placebo or a single administration of daratumumab on Day 0 combined with 40 mg oral prednisone.

FIGS. 12A-12B depict the effects of daratumumab on the percentage of CD38⁺ monocyte cells. CD38⁺ monocyte cells were enumerated via flow cytometry on PBMCs isolated from whole blood samples collected longitudinally from subjects treated with placebo or a single administration of daratumumab on Day 0. Shown are the percentages of CD38⁺ monocyte cells remaining after treatment compared to baseline within the parent CD45⁺CD3⁻CD19⁻CD56⁻CD14⁺CD16⁻CD38⁺ cell population. FIG. 12A The subjects were treated with placebo or a single administration of daratumumab on Day 0 without prednisone. FIG. 12B The subjects were treated with placebo or a single administration of daratumumab on Day 0 combined with 40 mg oral prednisone.

### DETAILED DESCRIPTION

A description of example embodiments follows.

While example embodiments have been particularly shown and described, it will be understood by those skilled in the art that various changes in form and details may be made therein without departing from the scope of the embodiments encompassed by the appended claims.

Various publications, articles and patents are cited or described in the background and throughout the specification; each of these references is herein incorporated by reference in its entirety. Discussion of documents, acts, materials, devices, articles or the like, which has been included in the present specification, is for the purpose of providing context to the present invention. Such discussion is not an admission that any or all of these matters form part of the prior art with respect to any inventions disclosed or claimed.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

Throughout this specification and the claims which follow, unless the context requires otherwise, the word “comprise,” and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The terms “subject” and “patient” can be used interchangeably herein. “Patient in need thereof” or “subject in need thereof” refers to a mammalian subject, preferably human, diagnosed with or suspected of having a disease, whom will be or has been administered an anti-CD38 antibody according to a method of the invention. “Patient in need thereof” or “subject in need thereof” includes those subjects already with the undesired physiological change or disease well as those subjects prone to have the physiological change or disease.

“An autoimmune disease” is a disease in which the immune system attacks healthy cells in the body by mistake. Diagnosis of an autoimmune disease can be done by a clinician according to clinical diagnostic testing, physical examination of the subject, or any other accepted method for diagnosing a subject with a particular autoimmune disease. “Patient suspected of having an autoimmune disease” or “subject suspected of having an autoimmune disease” is a subject that presents signs or symptoms indicative of an autoimmune disease that are discernable to a clinician and/or the subject, but whose suspected diagnosis has not been confirmed by clinical diagnostic testing, physical examination of the subject, or other accepted method for diagnosing a subject with the suspected autoimmune disease. The subject that may be treated also include those prone or susceptible to have an autoimmune disease, or those in which an autoimmune disease is to be prevented.

“CD38” refers to the human CD38 protein (synonyms: ADP-ribosyl cyclase 1, cADPr hydrolase 1, cyclic ADP-ribose hydrolase 1). Human CD38 has an amino acid sequence shown in GenBank accession number NP 001766 and in SEQ ID NO: 1. It is well known that CD38 is a single pass type II membrane protein with amino acid residues 1-21 representing the cytosolic domain, amino acid residues 22-42 representing the transmembrane domain, and residues 43-300 representing the extracellular domain of CD38.

SEQ ID NO: 1
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTSEI

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Antibodies” is meant in a broad sense and includes immunoglobulin molecules including monoclonal antibodies including murine, human, humanized and chimeric monoclonal antibodies, antigen-binding fragments, bispecific or multispecific antibodies, dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy (H) chains and two light (L) chains inter-connected by disulfide bonds as well as multimers thereof (for example IgM). Each heavy chain is comprised of a heavy chain variable region (VH) and a heavy chain constant region (comprised of domains CH1, hinge CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-terminus to carboxy-terminus in the following order: FR1, CDR1, FR2, CDR2, FR3, CDR3, and FR4.

“Complementarity determining regions (CDR)” are “antigen binding sites” in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, J Exp Med 132:211-50, 1970; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, Mol Biol 196:901-17, 1987). The International ImMunoGeneTics (IMGT) database (http://www_imgt_org) provides a standardized numbering and definition of antigen-binding sites. The correspondence between CDRs, HVs and IMGT delineations is described in Lefranc et al., Dev Comparat Immunol 27:55-77, 2003. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification.

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (x) and lambda (λ), based on the amino acid sequences of their constant domains.

“Antigen-binding fragment” refers to a portion of an immunoglobulin molecule that retains the antigen binding properties of the parental full length antibody. Exemplary antigen-binding fragments are as heavy chain complementarity determining regions (HCDR) 1, 2 and/or 3, light chain complementarity determining regions (LCDR) 1, 2 and/or 3, a heavy chain variable region (VH), or a light chain variable region (VL), Fab, F(ab′)2, Fd and Fv fragments as well as domain antibodies (dAb) consisting of either one VH domain or one VL domain. VH and VL domains may be linked together via a synthetic linker to form various types of single chain antibody designs in which the VH/VL domains pair intramolecularly, or intermolecularly in those cases when the VH and VL domains are expressed by separate chains, to form a monovalent antigen binding site, such as single chain Fv (scFv) or diabody; described for example in Int. Pat. Publ. No. WO1998/44001, Int. Pat. Publ. No. WO1988/01649; Int. Pat. Publ. No. WO1994/13804; Int. Pat. Publ. No. WO1992/01047.

“Monoclonal antibody” refers to an antibody population with single amino acid composition in each heavy and each light chain, except for possible well known alterations such as removal of C-terminal lysine from the antibody heavy chain. Monoclonal antibodies typically bind one antigenic epitope, except that multispecific monoclonal antibodies bind two or more distinct antigens or epitopes. Bispecific monoclonal antibodies bind two distinct antigenic epitopes. Monoclonal antibodies may have heterogeneous glycosylation within the antibody population. Monoclonal antibody may be monospecific or multispecific, or monovalent, bivalent or multivalent. A multispecific antibody, such as a bispecific antibody or a trispecific antibody is included in the term monoclonal antibody.

“Anti-CD38 antibody” refers to an antibody that binds CD38 with greater affinity than to other antigens. Typically, the antibody binds to CD38 with an equilibrium dissociation constant (KD) of about 1×10⁻⁸ M or less, for example about 1×10⁻⁹ M or less, about 1×10⁻¹⁰ M or less, about 1×10⁻¹¹ M or less, or about 1×10⁻¹²M or less, typically with a KD that is at least one hundred-fold less than its KD for binding to a non-specific antigen (e.g., BSA, casein). The KD may be measured using standard procedures. Antibodies that specifically bind CD38 may, however, have cross-reactivity to other related antigens, for example to the same antigen from other species (homologs), such as monkey, for example Macaca fascicularis (cynomolgus, cyno), Pan troglodytes (chimpanzee, chimp) or Callithrix jacchus (common marmoset, marmoset). In one embodiment, the anti-CD38 antibody binds to an epitope on human CD38 that includes amino acid residues 233-246 and 267-280 of CD38.

“Isolated antibody” refers to an antibody or an antigen-binding fragment thereof that is substantially free of other antibodies having different antigenic specificities (e.g., an isolated antibody specifically binding human CD38 is substantially free of antibodies that specifically bind antigens other than human CD38). In case of a bispecific antibody, the bispecific antibody specifically binds two antigens of interest, and is substantially free of antibodies that specifically bind antigens other that the two antigens of interest. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

“Humanized antibodies” refers to antibodies in which the antigen binding sites are derived from non-human species and the variable region frameworks are derived from human immunoglobulin sequences. Humanized antibodies may include intentionally introduced mutations in the framework regions so that the framework may not be an exact copy of expressed human immunoglobulin or germline gene sequences.

“Human antibodies” refers to antibodies having heavy and light chain variable regions in which both the framework and the antigen binding site are derived from sequences of human origin. If the antibody contains a constant region or a portion of the constant region, the constant region also is derived from sequences of human origin.

A human antibody comprises heavy or light chain variable regions that are derived from sequences of human origin if the variable regions of the antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci as described herein. A human antibody typically contains amino acid differences when compared to the human germline or rearranged immunoglobulin sequences due to, for example naturally occurring somatic mutations, intentional introduction of substitutions into the framework or antigen binding site and amino acid changes introduced during cloning and VDJ recombination in non-human animals. Typically, a human antibody is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene. In some cases, a human antibody may contain consensus framework sequences derived from human framework sequence analyses, for example as described in Knappik et al., J Mol Biol 296:57-86, 2000, or synthetic HCDR3 incorporated into human immunoglobulin gene libraries displayed on phage, for example as described in Shi et al., J Mol Biol 397:385-96, 2010 and Int. Pat. Publ. No. WO2009/085462.

Antibodies in which antigen binding sites are derived from a non-human species are not included in the definition of human antibody.

“Recombinant” includes antibodies and other proteins that are prepared, expressed, created or isolated by recombinant means.

“Variant” refers to a polypeptide or a polynucleotide that differs from a reference polypeptide or a reference polynucleotide by one or more modifications for example, substitutions, insertions or deletions.

“In combination with” means that two or more therapeutics are administered to a subject together in a mixture, concurrently as single agents or sequentially as single agents in any order.

“Pharmaceutical composition” refers to a product that results from combining an anti-CD38 antibody and a hyaluronidase and includes both fixed and non-fixed combinations. Pharmaceutical composition typically includes a pharmaceutically acceptable carrier. “Fixed combinations” refers to a single pharmaceutical composition comprising the anti-CD38 antibody and the hyaluronidase administered simultaneously in the form of a single entity or dosage. “Non-fixed combination” refers to separate pharmaceutical compositions of the anti-CD38 antibody and the hyaluronidase or unit dosage forms administered as separate entities either simultaneously, concurrently or sequentially with no specific intervening time limits, wherein such administration provides effective levels of the two compounds in the body of the subject.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

“Treat,” “treating” or “treatment” refers to therapeutic treatment wherein the objective is to slow down (lessen) an undesired physiological change or disease, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, disease remission (whether partial or total, whether detectable or undetectable), and prolonging survival as compared to expected survival if a subject was not receiving treatment or was receiving another treatment.

“Safe” as it relates to a composition, dose, dosage regimen, treatment or method with a therapeutic or a drug comprising an anti-CD38 antibody (such as daratumumab), refers to a favorable benefit:risk ratio with an acceptable frequency and/or acceptable severity of adverse events (AEs) and/or treatment-emergent adverse events (TEAEs) compared to the standard of care or to another comparator.

“A method of providing safe treatment” or “a method of providing safe administration” refers to a method of administration that is effective to provide the benefits of a therapeutic or pharmaceutical composition, without causing unacceptable adverse events, when administered to a subject.

“Adverse event” or “AE” refers to any untoward medical occurrence in a subject administered an antibody that specifically binds CD38, such as daratumumab. An AE does not necessarily have a causal relationship with the treatment. An AE can therefore be any unfavorable and unintended sign (including an abnormal finding), symptom, or disease temporally associated with the use of a medicinal (investigational or non investigational) product, whether or not related to the antibody that specifically binds CD38, such as daratumumab.

“Treatment emergent adverse events” (TEAE) as used herein takes its customary meaning as will be understood by a person skilled in the art of designing, conducting, or reviewing clinical trials and refers to an AE considered associated with the use of an antibody that specifically binds CD38 if the attribution is possible, probable, or very likely.

“Unacceptable adverse events” and “unacceptable adverse reaction” shall mean all harm or undesired outcomes associated with or caused by administration of a pharmaceutical composition or therapeutic, and the harm or undesired outcome reaches such a level of severity that a regulatory agency deems the pharmaceutical composition or therapeutic unacceptable for the proposed use. Examples of unacceptable adverse events or reactions when used in the context of subcutaneous administration of an anti-CD38 antibody include, but are not limited to, thrombocytopenia, neutropenia, severe systemic injection related reactions, and depletion of CD38⁺ cells to below certain specified levels.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the subject, reduction in a tumor burden, arrested or slowed growth of a tumor, and/or absence of metastasis of cancer cells to other locations in the body.

“Carrier” or “pharmaceutical carrier” refers to any excipient, diluent, buffer, stabilizer, or other material known in the art for pharmaceutical formulations.

“Corticosteroid” refers to a class of steroid hormones that are produced in the adrenal cortex or produced synthetically refers to dexamethasone, methylprednisolone, prednisolone and prednisone. Dexamethasone is marketed under the trade name DECARON®.

“Subcutaneous administration” refers to administration under the skin, in which a drug or therapeutic is injected into the tissue layer between the skin and muscle. Medication administered via subcutaneous administration is usually absorbed more slowly than if injected into a vein.

“Dosage” refers to the information of the amount of the therapeutic or the drug to be taken by the subject and the frequency of the number of times the therapeutic is to be taken by the subject. “Dose” refers to the amount or quantity of the therapeutic or the drug to be taken each time.

One aspect of the invention relates to a method of providing subcutaneous administration (e.g., safe subcutaneous administration) of an anti-CD38 antibody to a subject in need thereof, the method comprising subcutaneously administering to the subject a pharmaceutical composition comprising the anti-CD38 antibody and a pharmaceutically acceptable carrier.

The method is useful for treating a subject in need of anti-CD38 antibody therapy, such as a subject having an autoimmune disease, for example RA or SLE.

Another aspect of the invention relates to a method of providing treatment (e.g., providing safe treatment) of an autoimmune disease in a subject in need thereof, the method comprising subcutaneously administering to the subject a pharmaceutical composition comprising an anti-CD38 antibody and a pharmaceutically acceptable carrier.

In some embodiments, the subject is diagnosed with having an autoimmune disease.

In some embodiments, the subject is suspected of having an autoimmune disease.

In some embodiments, the autoimmune disease is responsive to treatment with a therapy that targets CD38.

In some embodiments, the subject in need thereof has or is suspected of having a disease selected from the group consisting of lupus, systemic lupus erythematosus, Sjogren's Syndrome, arthritis, rheumatoid arthritis, asthma, COPD, pelvic inflammatory disease, Alzheimer's Disease, inflammatory bowel disease, Crohn's disease, ulcerative colitis, Peyronie's Disease, celiac disease, gallbladder disease, Pilonidal disease, peritonitis, psoriasis, psoriatic arthritis, vasculitis, surgical adhesions, stroke, Type I Diabetes, Lyme disease, meningoencephalitis, autoimmune uveitis, multiple sclerosis, Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease, transplant rejection, heart disease including ischaemic diseases such as myocardial infarction as well as atherosclerosis, intravascular coagulation, bone resorption, osteoporosis, osteoarthritis, periodontitis and hypochlorhydia, infertility related to lack of fetal-maternal tolerance, vitiligo, myasthenia gravis or systemic sclerosis.

In some embodiments, the autoimmune disease is selected from arthritis, rheumatoid arthritis (RA), psoriatic arthritis, ankylosing spondylitis, ulcerative colitis, plaque psoriasis, systemic lupus erythematosus (SLE), lupus nephritis, antineutrophil cytoplasmic antibody (ANCA) associated vasculitis, myasthenia gravis, progressive multiple sclerosis, IgG4 related diseases, Sjogren's syndrome, immune thrombocytopenic purpura, transplant rejection, inflammatory bowel disease or Crohn's disease. IgG4-related disease is a chronic inflammatory condition characterized by tissue infiltration with lymphocytes and IgG4-secreting plasma cells and varying degrees of fibrosis (scarring).

In some embodiments of the invention, the autoimmune disease treated by the methods described herein is SLE.

In some embodiments, the subject is diagnosed with having RA.

In some embodiments, the subject is suspected of having RA.

In some embodiments, the subject is diagnosed with having SLE.

In some embodiments, the subject is suspected of having SLE.

In some embodiments, the subject has CD38 expression levels comparable to the level of CD38 expression in a healthy individual.

### Pharmaceutical Composition

In some embodiments, the pharmaceutical composition is a fixed combination.

In some embodiments, the pharmaceutical composition is a non-fixed combination.

Anti-CD38 antibodies used in the pharmaceutical compositions of the invention, may also be selected de novo from, e.g., a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-314, 1996; Sheets et al., PITAS (USA) 95:6157-6162, 1998; Hoogenboom and Winter, J Mol Biol 227:381, 1991; Marks et al., J Mol Biol 222:581, 1991). CD38 binding variable domains may be isolated from e.g., phage display libraries expressing antibody heavy and light chain variable regions as fusion proteins with bacteriophage pIX coat protein as described in Shi et al., J. Mol. Biol. 397:385-96, 2010 and Intl. Pat. Publ. No. WO09/085462). The antibody libraries may be screened for binding to human CD38 extracellular domain, the obtained positive clones further characterized, Fabs isolated from the clone lysates, and subsequently cloned as full length antibodies. Such phage display methods for isolating human antibodies are established in the art. See for example: U.S. Pat. Nos. 5,223,409, 5,403,484, 5,571,698, 5,427,908, 5,580,717, 5,969,108, 6,172,197, 5,885,793, 6,521,404, 6,544,731, 6,555,313, 6,582,915, and 6,593,081.

Antibodies may be evaluated for their competition with a reference antibody such as the anti-CD38 antibody comprising the VH of SEQ ID NO: 4 and the VL of SEQ ID NO: 5 for binding to CD38 using known in vitro methods. For example, Chinese Hamster Ovary (CHO) cells recombinantly expressing CD38 may be incubated with unlabeled reference antibody for 15 min at 4° C., followed by incubation with an excess of fluorescently labeled test antibody for 45 min at 4° C. After washing in PBS/BSA, fluorescence may be measured by flow cytometry using standard methods. In another method, extracellular portion of human CD38 may be coated on the surface of an ELISA plate. Excess of unlabeled reference antibody may be added for about 15 minutes and subsequently biotinylated test antibodies may be added. After washes in PBS/Tween, binding of the test biotinylated antibody may be detected using horseradish peroxidase (HRP)-conjugated streptavidine and the signal detected using standard methods. It is readily apparent that in the competition assays, the reference antibody may be labelled and the test antibody unlabeled. The test antibody competes with the reference antibody when the reference antibody inhibits binding of the test antibody, or the test antibody inhibits binding of the reference antibody by at least 80%, for example 81%, 82%, 83%, 84%, 85%, 86%, 87%, 88%, 89%, 90%, 91%, 92%, 93%, 94%, 95%, 96%, 97%, 98%, 99% or 100%. The epitope of the test antibody may further be defined for example by peptide mapping or hydrogen/deuterium protection assays using known methods, or by crystal structure determination.

In some embodiments, the anti-CD38 antibody binds at least to a region having a sequence of SKRNIQFSCKNIYR (SEQ ID NO: 2) and a region having a sequence of EKVQTLEAWVIHGG (SEQ ID NO: 3) of human CD38 (SEQ ID NO: 1). Antibodies binding to the region having the sequence of SKRNIQFSCKNIYR (SEQ ID NO: 2) and the region having the sequence of EKVQTLEAWVIHGG (SEQ ID NO: 3) of human CD38 (SEQ ID NO: 1) may be generated, for example, by immunizing mice with peptides having the amino acid sequences shown in SEQ ID NOs: 2 and 3 using standard methods and those described herein, and characterizing the obtained antibodies for binding to the peptides using for example ELISA or mutagenesis studies.

In some embodiments, the anti-CD38 antibody is a human monoclonal antibody (mAb) or an antigen binding fragment thereof.

In some embodiments, the anti-CD38 antibody competes for binding to CD38 with an antibody comprising a heavy chain variable region (VH) of SEQ ID NO: 4 and a light chain variable region (VL) of SEQ ID NO: 5.

In some embodiments, the anti-CD38 antibody comprises a VH that is 95%, 96%, 97%, 98%, 99% or 100% identical to the amino acid sequence of SEQ ID NO: 4 and a VL that is 95%, 96%, 97%, 98%, 99% or 100% identical to the amino acid sequence of SEQ ID NO: 5.

In some embodiments, the anti-CD38 antibody comprises the VH of SEQ ID NO: 4 and the VL of SEQ ID NO: 5.

In some embodiments, the anti-CD38 antibody comprises a HCDR1, a HCDR2, a HCDR3, of SEQ ID NOs: 6, 7 and 8, respectively, and a LCDR1, a LCDR2 and a LCDR3 of SEQ ID NOs: 9, 10 and 11, respectively.

In some embodiments, the anti-CD38 antibody comprises a heavy chain of SEQ ID NO: 12 and a light chain of SEQ ID NO: 13.

In some embodiments, the anti-CD38 antibody is a human mAb or an antigen binding fragment thereof of the immunoglobulin G1 kappa (IgG1K) subtype. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as G1m17, G1m3, Glm1, G1m2, G1m27 or G1m28.

In some embodiments, the anti-CD38 antibody is daratumumab. Daratumumab comprises the VH and the VL having amino acid sequences of SEQ ID NOs: 4 and 5, respectively; the HCDR1, the HCDR2 and the HCDR3 of SEQ ID NOs: 6, 7 and 8, respectively, the LCDR1, the LCDR2 and the LCDR3 of SEQ ID NOs: 9, 10 and 11, respectively; and is of IgG1/κ subtype and described in U.S. Pat. No. 7,829,693. Daratumumab comprises the heavy chain amino acid sequence of SEQ ID NO: 12 and the light chain amino acid sequence of SEQ ID NO: 13.

In some embodiments, daratumumab is DARZALEX® brand of daratumumab or a biosimilar of DARZALEX® brand of daratumumab. Daratumumab can be prepared by any method known in the art for preparing monoclonal antibodies including, but not limited to, hybridoma production. For example, daratumumab can be produced in a mammalian cell line (e.g., CHO cell line) using recombinant DNA technology. Daratumumab and methods of producing daratumumab are further described in, e.g., WO2006099875, U.S. Pat. No. 7,829,673, US2015246123, and de Weers et al. J. Immunol. 186: 1840-48, 2011, which are herein incorporated by reference.

Other exemplary anti-CD38 antibodies that may be used in the methods of the invention are:

mAb003 comprising the VH and the VL sequences of SEQ ID NOs: 14 and 15, respectively and described in U.S. Pat. No. 7,829,693. The VH and the VL of mAb003 may be expressed as IgG1/κ;

mAb024 comprising the VH and the VL sequences of SEQ ID NOs: 16 and 17, respectively, described in U.S. Pat. No. 7,829,693. The VH and the VL of mAb024 may be expressed as IgG1/κ;

MOR-202 (MOR-03087) comprising the VH and the VL sequences of SEQ ID NOs: 18 and 19, respectively, described in U.S. Pat. No. 8,088,896. The VH and the VL of MOR-202 may be expressed as IgG1/κ; or

Isatuximab; comprising the VH and the VL sequences of SEQ ID NOs: 20 and 21, respectively, described in U.S. Pat. No. 8,153,765. The VH and the VL of Isatuximab may be expressed as IgG1/κ.

Other exemplary anti-CD38 antibodies that may be used in the pharmaceutical compositions of the invention are those described in Int. Pat. Publ. No. WO05/103083, Intl. Pat. Publ. No. WO06/125640, Intl. Pat. Publ. No. WO07/042309, Intl. Pat. Publ. No. WO08/047242 or Intl. Pat. Publ. No. WO14/178820.

In some embodiments, the anti-CD38 antibody comprises a HCDR1, a HCDR2, a HCDR3, a LCDR1, a LCDR2, and a LCDR3 sequences of:

a. a VH of SEQ ID NO: 14 and a VL of SEQ ID NO: 15;

b. a VH of SEQ ID NO: 16 and a VL of SEQ ID NO: 17;

c. a VH of SEQ ID NO: 18 and a VL of SEQ ID NO: 19; or

d. a VH of SEQ ID NO: 20 and a VL of SEQ ID NO: 21.

In some embodiments, the anti-CD38 antibody comprises

a. a VH of SEQ ID NO: 14 and a VL of SEQ ID NO: 15;

b. a VH of SEQ ID NO: 16 and a VL of SEQ ID NO: 17;

c. a VH of SEQ ID NO: 18 and a VL of SEQ ID NO: 19; or

d. a VH of SEQ ID NO: 20 and a VL of SEQ ID NO: 21.

SEQ ID NO: 2
SKRNIQFSCKNIYR
SEQ ID NO: 3
EKVQTLEAWVIHGG
SEQ ID NO: 4
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYYCAKDK
ILWFGEPVFDYWGQGTLVTVSS
SEQ ID NO: 5
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIK
SEQ ID NO: 6
SFAMS
SEQ ID NO: 7
AISGSGGGTYYADSVKG
SEQ ID NO: 8
DKILWFGEPVFDY
SEQ ID NO: 9
RASQSVSSYLA
SEQ ID NO: 10
DASNRAT
SEQ ID NO: 11
QQRSNWPPTF
SEQ ID NO: 12
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
SEQ ID NO: 13
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 14
QVQLVQSGAEVKKPGSSVKVSCKASGGTFSSYAFSWVRQAPGQGLEWMGR
VIPFLGIANSAQKFQGRVTITADKSTSTAYMDLSSLRSEDTAVYYCARDD
IAALGPFDYWGQGTLVTVSSAS
SEQ ID NO: 15
DIQMTQSPSSLSASVGDRVTITCRASQGISSWLAWYQQKPEKAPKSLIYA
ASSLQSGVPSRFSGSGSGTDFTLTISSLQPEDFATYYCQQYNSYPRTFGQ
GTKVEIK
SEQ ID NO: 16
EVQLVQSGAEVKKPGESLKISCKGSGYSFSNYWIGWVRQMPGKGLEWMGI
IYPHDSDARYSPSFQGQVTFSADKSISTAYLQWSSLKASDTAMYYCARHV
GWGSRYWYFDLWGRGTLVTVSS
SEQ ID NO: 17
EIVLTQSPATLSLSPGERATLSCRASQSVSSYLAWYQQKPGQAPGLLIYD
ASNRASGIPARFSGSGSGTDFTLTISSLEPEDFAVYYCQQRSNWPLTFGG
GTKVEIK
SEQ ID NO: 18
QVQLVESGGGLVQPGGSLRLSCAASGFTFSSYYMNWVRQAPGKGLEWVSG
ISGDPSNTYYADSVKGRFTISRDNSKNTLYLQMNSLRAEDTAVYYCARDL
PLVYTGFAYWGQGTLVTVSS
SEQ ID NO: 19
DIELTQPPSVSVAPGQTARISCSGDNLRHYYVYWYQQKPGQAPVLVIYGD
SKRPSGIPERFSGSNSGNTATLTISGTQAEDEADYYCQTYTGGASLVFGG
GTKLTVLGQ
SEQ ID NO 20:
QVQLVQSGAEVKAKPGTSVKLSCKASGYTFTDYWMQWVKQRPGQGLEWIG
TIYPGDGDTGYAQKFQGKATLTADKSSKTVYMHLSSLASEDSAVYYCARG
DYYGSNSLDYWGQGTSVTVSS
SEQ ID NO: 21:
DIVMTQSHLSMSTSLGDPVSITCKASQDVSTVVAWYQQKPGQSPRRLIYS
ASYRYIGVPDRFTGSGAGTDFTFTISSVQAEDLAVYYCQQHYSPPYTFGG
GTKLEIK

In some embodiments, a total dosage of the anti-CD38 antibody is from about 10 mg to about 600 mg per administration.

In some embodiments, a total dosage of the anti-CD38 antibody is from about 10 mg to about 550 mg per administration.

In some embodiments, a total dosage of the anti-CD38 antibody is from about 15 mg to about 550 mg per administration.

In some embodiments, a total dosage of the anti-CD38 antibody is from about 15 mg to about 500 mg per administration.

In some embodiments, a total dosage of the anti-CD38 antibody is from about 25 mg to about 500 mg per administration.

In some embodiments, a total dosage of the anti-CD38 antibody is from about 25 mg to about 450 mg per administration.

In some embodiments, a total dosage of the anti-CD38 antibody is from about 40 mg to about 450 mg per administration.

In some embodiments, a total dosage of the anti-CD38 antibody is from about 40 mg to about 400 mg per administration.

In some embodiments, a total dosage of the anti-CD38 antibody is from about 60 mg to about 400 mg per administration.

In some embodiments, a total dosage of the anti-CD38 antibody is from about 60 mg to about 350 mg per administration.

In some embodiments, a total dosage of the anti-CD38 antibody is from about 100 mg to about 350 mg per administration.

In some embodiments, a total dosage of the anti-CD38 antibody is from about 100 mg to about 300 mg per administration.

In some embodiments, a total dosage of the anti-CD38 antibody is from about 150 mg to about 300 mg per administration.

In some embodiments, a total dosage of the anti-CD38 antibody is from about 150 mg to about 250 mg per administration.

In some embodiments, a total dosage of the anti-CD38 antibody is from about 200 mg to about 250 mg per administration.

In some embodiments, a total dosage of the anti-CD38 antibody is about 10 mg, about 20 mg, about 25 mg, about 30 mg, about 40 mg, about 50 mg, about 60 mg, about 70 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg or about 600 mg, per administration.

In some embodiments, a total dosage of the anti-CD38 antibody is about 200 mg per administration.

In some embodiments, a total dosage of the anti-CD38 antibody is about 350 mg per administration.

In some embodiments, a total dosage of the anti-CD38 antibody is about 600 mg per administration.

In some embodiments, a total dosage of the anti-CD38 antibody is from about 10 mg to about 2,400 mg per administration.

In some embodiments, a total dosage of the anti-CD38 antibody is from about 10 mg to about 2,000 mg per administration.

In some embodiments, a total dosage of the anti-CD38 antibody is from about 20 mg to about 2,000 mg per administration.

In some embodiments, a total dosage of the anti-CD38 antibody is from about 20 mg to about 1,500 mg per administration.

In some embodiments, a total dosage of the anti-CD38 antibody is from about 50 mg to about 1,500 mg per administration.

In some embodiments, a total dosage of the anti-CD38 antibody is from about 50 mg to about 1,000 mg per administration.

In some embodiments, a total dosage of the anti-CD38 antibody is from about 100 mg to about 1,000 mg per administration.

In some embodiments, a total dosage of the anti-CD38 antibody is from about 100 mg to about 500 mg per administration.

In some embodiments, a total dosage of the anti-CD38 antibody is from about 200 mg to about 500 mg per administration.

In some embodiments, a total dosage of the anti-CD38 antibody is about 700 mg, about 800 mg, about 900 mg, about 1,000 mg, about 1,100 mg, about 1,200 mg, about 1,300 mg, about 1,400 mg, about 1,500 mg, about 1,600 mg, about 1,700 mg, about 1,800 mg, about 1,900 mg, about 2,000 mg, about 2,100 mg, about 2,200 mg, about 2,300 mg, or about 2,400 mg, per administration.

In some embodiments, a total dosage of the anti-CD38 antibody is about 1,000 mg per administration.

In some embodiments, a total dosage of the anti-CD38 antibody is about 1,500 mg per administration.

In some embodiments, a total dosage of the anti-CD38 antibody is about 2,000 mg per administration.

In some embodiments, a total dosage of the anti-CD38 antibody is about 2,400 mg per administration.

The dose given to the subject is sufficient to alleviate or at least partially arrest the disease being treated (“therapeutically effective amount”) and may be sometimes 0.005 mg to about 100 mg/kg, e.g., about 0.05 mg to about 30 mg/kg or about 5 mg to about 25 mg/kg, or about 4 mg/kg, about 8 mg/kg, about 16 mg/kg or about 24 mg/kg, or for example about 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

The concentration of the anti-CD38 antibody included in the pharmaceutical compositions can vary.

In some embodiments, the pharmaceutical composition comprises from about 1 mg/mL to about 180 mg/mL of the anti-CD38 antibody.

In some embodiments, the pharmaceutical composition comprises from about 2 mg/mL to about 180 mg/mL of the anti-CD38 antibody.

In some embodiments, the pharmaceutical composition comprises from about 2 mg/mL to about 175 mg/mL of the anti-CD38 antibody.

In some embodiments, the pharmaceutical composition comprises from about 5 mg/mL to about 175 mg/mL of the anti-CD38 antibody.

In some embodiments, the pharmaceutical composition comprises from about 5 mg/mL to about 170 mg/mL of the anti-CD38 antibody.

In some embodiments, the pharmaceutical composition comprises from about 10 mg/mL to about 170 mg/mL of the anti-CD38 antibody.

In some embodiments, the pharmaceutical composition comprises from about 10 mg/mL to about 165 mg/mL of the anti-CD38 antibody.

In some embodiments, the pharmaceutical composition comprises from about 20 mg/mL to about 165 mg/mL of the anti-CD38 antibody.

In some embodiments, the pharmaceutical composition comprises from about 20 mg/mL to about 160 mg/mL of the anti-CD38 antibody.

In some embodiments, the pharmaceutical composition comprises from about 40 mg/mL to about 160 mg/mL of the anti-CD38 antibody.

In some embodiments, the pharmaceutical composition comprises from about 40 mg/mL to about 155 mg/mL of the anti-CD38 antibody.

In some embodiments, the pharmaceutical composition comprises from about 60 mg/mL to about 155 mg/mL of the anti-CD38 antibody.

In some embodiments, the pharmaceutical composition comprises from about 60 mg/mL to about 150 mg/mL of the anti-CD38 antibody.

In some embodiments, the pharmaceutical composition comprises from about 80 mg/mL to about 150 mg/mL of the anti-CD38 antibody.

In some embodiments, the pharmaceutical composition comprises from about 80 mg/mL to about 145 mg/mL of the anti-CD38 antibody.

In some embodiments, the pharmaceutical composition comprises from about 100 mg/mL to about 145 mg/mL of the anti-CD38 antibody.

In some embodiments, the pharmaceutical composition comprises from about 100 mg/mL to about 140 mg/mL of the anti-CD38 antibody.

In some embodiments, the pharmaceutical composition comprises from about 110 mg/mL to about 140 mg/mL of the anti-CD38 antibody.

In some embodiments, the pharmaceutical composition comprises from about 110 mg/mL to about 135 mg/mL of the anti-CD38 antibody.

In some embodiments, the pharmaceutical composition comprises from about 115 mg/mL to about 135 mg/mL of the anti-CD38 antibody.

In some embodiments, the pharmaceutical composition comprises from about 115 mg/mL to about 130 mg/mL of the anti-CD38 antibody.

In some embodiments, the pharmaceutical composition comprises from about 120 mg/mL to about 130 mg/mL of the anti-CD38 antibody.

In some embodiments, the pharmaceutical composition comprises about 1 mg/mL, about 2 mg/mL, about 5 mg/mL, about 10 mg/mL, about 15 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, or about 180 mg/mL of the anti-CD38 antibody.

In some embodiments, the pharmaceutical composition comprises about 100 mg/mL of the anti-CD38 antibody.

In some embodiments, the pharmaceutical composition comprises about 120 mg/mL of the anti-CD38 antibody.

In some embodiments, the pharmaceutical composition comprises about 140 mg/mL of the anti-CD38 antibody.

The antibodies that specifically bind CD38 in the methods of the invention may be lyophilized for storage and reconstituted in a suitable carrier prior to use. This technique has been shown to be effective with conventional protein preparations and well known lyophilization and reconstitution techniques can be employed.

In some embodiments, the anti-CD38 antibody is subcutaneously administered with recombinant human hyaluronidase (e.g., rHuPH20). Hyaluronidase is an enzyme that degrades hyaluronic acid (EC 3.2.1.35) and lowers the viscosity of hyaluronan in the extracellular matrix, thereby increasing tissue permeability.

For subcutaneous administration of larger volumes, as typically needed for antibody solutions and compositions, the extracellular matrix of the subcutaneous tissue presents a problem. The space outside adipocytes in the hypodermis is not a fluid, but rather a solid extracellular matrix of collagenous fibrils embedded within a glycosaminoglycan-rich viscoelastic gel that buffers convective forces. The extracellular matrix limits the volume of drug that can be injected at a single site, as well as the rate and amount that reach the vascular compartment. Thus, co-formulation or co-administration of an antibody with recombinant human hyaluronidase, such as rHuPH20, has allowed for increased injection volumes and bioavailability from subcutaneous injection.

Enzymatic activity of hyaluronidase, including rHuPH20 can be defined by units per mL (U/mL) or by total enzyme activity in a particular formulation (U). The standard definition for one unit (U) of enzyme activity is the amount of enzyme that catalyzes the reaction of 1 nmol of substrate per minute.

In some embodiments, the pharmaceutical composition comprises from about 50 U/mL to about 5,000 U/mL of the hyaluronidase.

In some embodiments, the pharmaceutical composition comprises from about 500 U/mL to about 5,000 U/mL of the hyaluronidase.

In some embodiments, the pharmaceutical composition comprises from about 1,000 U/mL to about 5,000 U/mL of the hyaluronidase.

In some embodiments, the pharmaceutical composition comprises from about 2,000 U/mL to about 5,000 U/mL of the hyaluronidase.

In some embodiments, the pharmaceutical composition comprises from about 50 U/mL to about 2,000 U/mL of the hyaluronidase.

In some embodiments, the pharmaceutical composition comprises from about 500 U/mL to about 2,000 U/mL of the hyaluronidase.

In some embodiments, the pharmaceutical composition comprises from about 1,000 U/mL to about 2,000 U/mL of the hyaluronidase.

In some embodiments, the pharmaceutical composition comprises about 500 U/mL, about 600 U/mL, about 700 U/mL, about 800 U/mL, about 900 U/mL, about 1,000 U/mL, about 1,100 U/mL, about 1,200 U/mL, about 1,300 U/mL, about 1,400 U/mL, about 1,500 U/mL, about 1,600 U/mL, about 1,700 U/mL, about 1,800 U/mL, about 1,900 U/mL, about 2,000 U/mL, about 2,100 U/mL, about 2,200 U/mL, about 2,300 U/mL, about 2,400 U/mL, about 2,500 U/mL, about 2,600 U/mL, about 2,700 U/mL, about 2,800 U/mL, about 2,900 U/mL, about 3,000 U/mL, about 3,100 U/mL, about 3,200 U/mL, about 3,300 U/mL, about 3,400 U/mL, about 3,500 U/mL, about 3,600 U/mL, about 3,700 U/mL, about 3,800 U/mL, about 3,900 U/mL, about 4,000 U/mL, about 4,100 U/mL, about 4,200 U/mL, about 4,300 U/mL, about 4,400 U/mL, about 4,500 U/mL, about 4,600 U/mL, about 4,700 U/mL, about 4,800 U/mL, about 4,900 U/mL or about 5,000 U/mL of the hyaluronidase.

In some embodiments, the pharmaceutical composition comprises about 500 U/mL of the hyaluronidase.

In some embodiments, the pharmaceutical composition comprises about 2,000 U/mL of the hyaluronidase.

In some embodiments, the pharmaceutical composition comprises about 5,000 U/mL of the hyaluronidase.

In some embodiments, the pharmaceutical composition comprises from about 750 U to about 75,000 U of the hyaluronidase.

In some embodiments, the pharmaceutical composition comprises from about 7,500 U to about 45,000 U of the hyaluronidase.

In some embodiments, the pharmaceutical composition comprises from about 30,000 U to about 45,000 U of the hyaluronidase.

In some embodiments, the pharmaceutical composition comprises about 7,500 U, about 8,000 U, about 8,500 U, about 9,000 U, about 10,000 U, about 15,000 U, about 20,000 U, about 21,000 U, about 22,000 U, about 23,000 U, about 24,000 U, about 25,000 U, about 26,000 U, about 27,000 U, about 28,000 U, about 29,000 U, about 30,000 U, about 31,000 U, about 32,000 U, about 33,000 U, about 34,000 U, about 35,000 U, about 36,000 U, about 37,000 U, about 38,000 U, about 39,000 U, about 40,000 U, about 41,000 U, about 42,000 U, about 43,000 U, about 44,000 U, about 45,000 U, about 46,000 U, about 47,000 U, about 48,000 U, about 49,000 U, about 50,000 U, about 55,000 U, about 60,000 U, about 65,000 U, about 70,000 U or about 75,000 U of the hyaluronidase.

In some embodiments, the pharmaceutical composition comprises about 5,000 mg of the anti-CD38 antibody and about 30,000 U of the hyaluronidase.

In some embodiments, the pharmaceutical composition comprises about 5,000 mg of the anti-CD38 antibody and about 45,000 U of the hyaluronidase.

In some embodiments, the pharmaceutical composition comprises about 3,000 mg of the anti-CD38 antibody and about 30,000 U of the hyaluronidase.

In some embodiments, the pharmaceutical composition comprises about 3,000 mg of the anti-CD38 antibody and about 45,000 U of the hyaluronidase.

In some embodiments, the pharmaceutical composition comprises about 2,800 mg of the anti-CD38 antibody and about 30,000 U of the hyaluronidase.

In some embodiments, the pharmaceutical composition comprises about 2,800 mg of the anti-CD38 antibody and about 45,000 U of the hyaluronidase.

In some embodiments, the pharmaceutical composition comprises about 2,600 mg of the anti-CD38 antibody and about 30,000 U of the hyaluronidase.

In some embodiments, the pharmaceutical composition comprises about 2,600 mg of the anti-CD38 antibody and about 45,000 U of the hyaluronidase.

In some embodiments, the pharmaceutical composition comprises about 2,400 mg of the anti-CD38 antibody and about 30,000 U of the hyaluronidase.

In some embodiments, the pharmaceutical composition comprises about 2,400 mg of the anti-CD38 antibody and about 45,000 U of the hyaluronidase.

In some embodiments, the pharmaceutical composition comprises about 2,200 mg of the anti-CD38 antibody and about 30,000 U of the hyaluronidase.

In some embodiments, the pharmaceutical composition comprises about 2,200 mg of the anti-CD38 antibody and about 45,000 U of the hyaluronidase.

In some embodiments, the pharmaceutical composition comprises about 2,000 mg of the anti-CD38 antibody and about 30,000 U of the hyaluronidase.

In some embodiments, the pharmaceutical composition comprises about 2,000 mg of the anti-CD38 antibody and about 45,000 U of the hyaluronidase.

In some embodiments, the pharmaceutical composition comprises about 1,800 mg of the anti-CD38 antibody and about 30,000 U of the hyaluronidase.

In some embodiments, the pharmaceutical composition comprises about 1,800 mg of the anti-CD38 antibody and about 45,000 U of the hyaluronidase.

In some embodiments, the pharmaceutical composition comprises about 1,600 mg of the anti-CD38 antibody and about 30,000 U of the hyaluronidase.

In some embodiments, the pharmaceutical composition comprises about 1,600 mg of the anti-CD38 antibody and about 45,000 U of the hyaluronidase.

In some embodiments, the hyaluronidase is rHuPH20 having the amino acid sequence of SEQ ID NO: 22.

rHuPH20 is a recombinant hyaluronidase (HYLENEX® recombinant) and is described in Int. Pat. Publ. No. WO2004/078140.

SEQ ID NO: 22
MGVLKFKHIFFRSFVKSSGVSQIVFTFLLIPCCLTLNFRAPPVIPNVPFL
WAWNAPSEFCLGKFDEPLDMSLFSFIGSPRINATGQGVTIFYVDRLGYYP
YIDSITGVTVNGGIPQKISLQDHLDKAKKDITFYMPVDNLGMAVIDWEEW
RPTWARNWKPKDVYKNRSIELVQQQNVQLSLTEATEKAKQEFEKAGKDFL
VETIKLGKLLRPNHLWGYYLFPDCYNHHYKKPGYNGSCFNVEIKRNDDLS
WLWNESTALYPSIYLNTQQSPVAATLYVRNRVREAIRVSKIPDAKSPLPV
FAYTRIVFTDQVLKFLSQDELVYTFGETVALGASGIVIWGTLSIMRSMKS
CLLLDNYMETILNPYIINVTLAAKMCSQVLCQEQGVCIRKNWNSSDYLHL
NPDNFAIQLEKGGKFTVRGKPTLEDLEQFSEKFYCSCYSTLSCKEKADVK
DTDAVDVCIADGVCIDAFLKPPMETEEPQIFYNASPSTLSATMFIVSILF
LIISSVAS

In some embodiments, the anti-CD38 antibody is subcutaneously administered without recombinant human hyaluronidase.

In some embodiments, a lower volume of the anti-CD38 antibody is administered to a subject diagnosed with or suspected of having an autoimmune disease compared to a subject diagnosed with or suspected of having cancer. The reduced volume is, at least in part, due to a reduced dose of the anti-CD38 antibody that can be effectively administered subcutaneously to a subject diagnosed with or suspected of having an autoimmune disease without the need for co-formulating the anti-CD38 antibody with recombinant human hyaluronidase. Moreover, the reduced volume (due in part to the reduced dose) allows for reduced administration time, which may lead to increased plasma drug concentrations and reduced infusion related reactions (IRRs) associated with intravenous administration.

Pharmaceutical compositions suitable for use in the methods of the invention are formulated for subcutaneous administration. Examples of formulations suitable for subcutaneous administration include, but are not limited to, solutions, suspensions, emulsions, and dry products that can be dissolved or suspended in a pharmaceutically acceptable carrier for injection.

In some embodiments, the pharmaceutical composition comprising the anti-CD38 antibody for use in the methods of the invention is formulated as a solution.

Pharmaceutical compositions suitable for use in the methods of the invention further comprise one or more pharmaceutically acceptable carriers, such as those widely employed in the art of drug manufacturing, and particularly antibody drug manufacturing. Pharmaceutically acceptable carriers in particular are non-toxic and should not interfere with the efficacy of the active ingredient. The carrier may be diluent, adjuvant, excipient, or vehicle with which the antibodies that specifically bind CD38 are administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies that specifically bind CD38 in such pharmaceutical formulation may vary widely, i.e., from less than about 0.5%, usually to at least about 1% to as much as 15 or 20%, 25%, 30%, 35%, 40%, 45% or 50% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, Pa. 2006, Part 5, Pharmaceutical Manufacturing pp 691-1092, see especially pp. 958-89.

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

The amounts of pharmaceutically acceptable carrier(s) in the pharmaceutical compositions may be determined experimentally based on the activities of the carrier(s) and the desired characteristics of the formulation, such as stability and/or minimal oxidation.

In some embodiments, the pharmaceutical composition comprises histidine at a concentration of from about 1 mM to about 50 mM.

In some embodiments, the pharmaceutical composition comprises histidine at a concentration of from about 2 mM to about 50 mM.

In some embodiments, the pharmaceutical composition comprises histidine at a concentration of from about 2 mM to about 40 mM.

In some embodiments, the pharmaceutical composition comprises histidine at a concentration of about 5 mM to about 50 mM.

In some embodiments, the pharmaceutical composition comprises histidine at a concentration of from about 5 mM to about 40 mM.

In some embodiments, the pharmaceutical composition comprises histidine at a concentration of from about 5 mM to about 30 mM.

In some embodiments, the pharmaceutical composition comprises histidine at a concentration of from about 5 mM to about 20 mM.

In some embodiments, the pharmaceutical composition comprises histidine at a concentration of from about 5 mM to about 15 mM.

In some embodiments, the pharmaceutical composition comprises histidine at a concentration of from about 5 mM to about 10 mM.

In some embodiments, the pharmaceutical composition comprises histidine at a concentration of from about 10 mM to about 30 mM.

In some embodiments, the pharmaceutical composition comprises histidine at a concentration of from about 10 mM to about 20 mM.

In some embodiments, the pharmaceutical composition comprises histidine at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM or about 50 mM.

In some embodiments, the pharmaceutical composition comprises histidine at a concentration of about 5 mM.

In some embodiments, the pharmaceutical composition comprises histidine at a concentration of about 10 mM.

In some embodiments, the pharmaceutical composition comprises histidine at a concentration of about 15 mM.

In some embodiments, the pharmaceutical composition comprises histidine at a concentration of about 20 mM.

In some embodiments, the pharmaceutical composition comprises methionine at a concentration of from about 0.1 mg/mL to about 5 mg/mL.

In some embodiments, the pharmaceutical composition comprises methionine at a concentration of from about 0.1 mg/mL to about 2.5 mg/mL.

In some embodiments, the pharmaceutical composition comprises methionine at a concentration of from about 0.2 mg/mL to about 5 mg/mL.

In some embodiments, the pharmaceutical composition comprises methionine at a concentration of from about 0.2 mg/mL to about 4 mg/mL.

In some embodiments, the pharmaceutical composition comprises methionine at a concentration of from about 0.5 mg/mL to about 4 mg/mL.

In some embodiments, the pharmaceutical composition comprises methionine at a concentration of from about 0.5 mg/mL to about 3 mg/mL.

In some embodiments, the pharmaceutical composition comprises methionine at a concentration of from about 1 mg/mL to about 3 mg/mL.

In some embodiments, the pharmaceutical composition comprises methionine at a concentration of from about 1 mg/mL to about 2 mg/mL.

In some embodiments, the pharmaceutical composition comprises methionine at a concentration of about 0.5 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, about 4 mg/mL, about 4.1 mg/mL, about 4.2 mg/mL, about 4.3 mg/mL, about 4.4 mg/mL, about 4.5 mg/mL, about 4.6 mg/mL, about 4.7 mg/mL, about 4.8 mg/mL, about 4.9 mg/mL or about 5 mg/mL.

In some embodiments, the pharmaceutical composition comprises acetic acid.

In some embodiments, the pharmaceutical composition comprises acetic acid at a concentration of from about 1 mM to about 50 mM.

In some embodiments, the pharmaceutical composition comprises acetic acid at a concentration of from about 2 mM to about 50 mM.

In some embodiments, the pharmaceutical composition comprises acetic acid at a concentration of from about 2 mM to about 45 mM.

In some embodiments, the pharmaceutical composition comprises acetic acid at a concentration of from about 5 mM to about 45 mM.

In some embodiments, the pharmaceutical composition comprises acetic acid at a concentration of from about 5 mM to about 40 mM.

In some embodiments, the pharmaceutical composition comprises acetic acid at a concentration of from about 10 mM to about 40 mM.

In some embodiments, the pharmaceutical composition comprises acetic acid at a concentration of from about 10 mM to about 35 mM.

In some embodiments, the pharmaceutical composition comprises acetic acid at a concentration of from about 15 mM to about 35 mM.

In some embodiments, the pharmaceutical composition comprises acetic acid at a concentration of from about 15 mM to about 30 mM.

In some embodiments, the pharmaceutical composition comprises acetic acid at a concentration of from about 20 mM to about 30 mM.

In some embodiments, the pharmaceutical composition comprises acetic acid at a concentration of about 10 mM, about 15 mM, about 20 mM, about 25 mM, about 30 mM, about 35 mM, about 40 mM, about 45 mM or about 50 mM.

In some embodiments, the pharmaceutical composition comprises acetic acid at a concentration of about 25 mM.

In some embodiments, the pharmaceutical composition comprises NaCl.

In some embodiments, the pharmaceutical composition comprises NaCl at a concentration of from about 20 mM to about 100 mM.

In some embodiments, the pharmaceutical composition comprises NaCl at a concentration of from about 20 mM to about 90 mM.

In some embodiments, the pharmaceutical composition comprises NaCl at a concentration of from about 30 mM to about 90 mM.

In some embodiments, the pharmaceutical composition comprises NaCl at a concentration of from about 30 mM to about 80 mM.

In some embodiments, the pharmaceutical composition comprises NaCl at a concentration of from about 40 mM to about 80 mM.

In some embodiments, the pharmaceutical composition comprises NaCl at a concentration of from about 40 mM to about 70 mM.

In some embodiments, the pharmaceutical composition comprises NaCl at a concentration of from about 50 mM to about 70 mM.

In some embodiments, the pharmaceutical composition comprises NaCl at a concentration of about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM.

In some embodiments, the pharmaceutical composition comprises NaCl at a concentration of about 60 mM.

In some embodiments, the pharmaceutical composition comprises sodium acetate at a concentration of from about 10 mM to about 50 mM.

In some embodiments, the pharmaceutical composition comprises sodium acetate at a concentration of from about 15 mM to about 50 mM.

In some embodiments, the pharmaceutical composition comprises sodium acetate at a concentration of from about 15 mM to about 45 mM.

In some embodiments, the pharmaceutical composition comprises sodium acetate at a concentration of from about 20 mM to about 45 mM.

In some embodiments, the pharmaceutical composition comprises sodium acetate at a concentration of about 20 mM to about 40 mM.

In some embodiments, the pharmaceutical composition comprises sodium acetate at a concentration of from about 25 mM to about 40 mM.

In some embodiments, the pharmaceutical composition comprises sodium acetate at a concentration of from about 25 mM to about 35 mM.

In some embodiments, the pharmaceutical composition comprises sodium acetate at a concentration of about 10 mM, about 15 mM, about 20 mM, about 25 mM, about 30 mM, about 35 mM, about 40 mM, about 45 mM, or about 50 mM.

In some embodiments, the pharmaceutical composition comprises sodium acetate at a concentration of about 30 mM.

In some embodiments, the pharmaceutical composition comprises saccharide.

In some embodiments, saccharide is sucrose.

In some embodiments, saccharide is sorbitol.

In some embodiments, saccharide is mannitol.

In some embodiments, the pharmaceutical composition comprises saccharide at a concentration of from about 50 mM to about 500 mM.

In some embodiments, the pharmaceutical composition comprises saccharide at a concentration of from about 50 mM to about 450 mM.

In some embodiments, the pharmaceutical composition comprises saccharide at a concentration of from about 50 mM to about 400 mM.

In some embodiments, the pharmaceutical composition comprises saccharide at a concentration of from about 50 mM to about 350 mM.

In some embodiments, the pharmaceutical composition comprises saccharide at a concentration of from about 60 mM to about 500 mM.

In some embodiments, the pharmaceutical composition comprises saccharide at a concentration of from about 60 mM to about 450 mM.

In some embodiments, the pharmaceutical composition comprises saccharide at a concentration of from about 70 mM to about 450 mM.

In some embodiments, the pharmaceutical composition comprises saccharide at a concentration of from about 70 mM to about 400 mM.

In some embodiments, the pharmaceutical composition comprises saccharide at a concentration of from about 80 mM to about 400 mM.

In some embodiments, the pharmaceutical composition comprises saccharide at a concentration of from about 80 mM to about 350 mM.

In some embodiments, the pharmaceutical composition comprises saccharide at a concentration of from about 90 mM to about 350 mM.

In some embodiments, the pharmaceutical composition comprises saccharide at a concentration of from about 90 mM to about 300 mM.

In some embodiments, the pharmaceutical composition comprises saccharide at a concentration of from about 100 mM to about 350 mM.

In some embodiments, the pharmaceutical composition comprises saccharide at a concentration of from about 100 mM to about 300 mM.

In some embodiments, the pharmaceutical composition comprises saccharide at a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, the pharmaceutical composition comprises mannitol.

In some embodiments, the pharmaceutical composition comprises mannitol at a concentration of from about 100 mM to about 180 mM.

In some embodiments, the pharmaceutical composition comprises mannitol at a concentration of from about 105 mM to about 180 mM.

In some embodiments, the pharmaceutical composition comprises mannitol at a concentration of from about 105 mM to about 175 mM.

In some embodiments, the pharmaceutical composition comprises mannitol at a concentration of from about 110 mM to about 175 mM.

In some embodiments, the pharmaceutical composition comprises mannitol at a concentration of from about 110 mM to about 170 mM.

In some embodiments, the pharmaceutical composition comprises mannitol at a concentration of from about 115 mM to about 170 mM.

In some embodiments, the pharmaceutical composition comprises mannitol at a concentration of from about 115 mM to about 165 mM.

In some embodiments, the pharmaceutical composition comprises mannitol at a concentration of from about 120 mM to about 165 mM.

In some embodiments, the pharmaceutical composition comprises mannitol at a concentration of from about 120 mM to about 160 mM.

In some embodiments, the pharmaceutical composition comprises mannitol at a concentration of from about 125 mM to about 160 mM.

In some embodiments, the pharmaceutical composition comprises mannitol at a concentration of from about 125 mM to about 155 mM.

In some embodiments, the pharmaceutical composition comprises mannitol at a concentration of from about 130 mM to about 155 mM.

In some embodiments, the pharmaceutical composition comprises mannitol at a concentration of from about 130 mM to about 150 mM.

In some embodiments, the pharmaceutical composition comprises mannitol at a concentration of from about 140 mM to about 180 mM.

In some embodiments, the pharmaceutical composition comprises mannitol at a concentration of about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM or about 180 mM.

In some embodiments, the pharmaceutical composition comprises mannitol at a concentration of about 140 mM.

In some embodiments, the pharmaceutical composition comprises sorbitol at a concentration of from about 50 mM to about 500 mM.

In some embodiments, the pharmaceutical composition comprises sorbitol at a concentration of from about 50 mM to about 450 mM.

In some embodiments, the pharmaceutical composition comprises sorbitol at a concentration of from about 50 mM to about 400 mM.

In some embodiments, the pharmaceutical composition comprises sorbitol at a concentration of from about 50 mM to about 350 mM.

In some embodiments, the pharmaceutical composition comprises sorbitol at a concentration of from about 100 mM to about 350 mM.

In some embodiments, the pharmaceutical composition comprises sorbitol at a concentration of from about 100 mM to about 300 mM.

In some embodiments, the pharmaceutical composition comprises sorbitol at a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, the pharmaceutical composition comprises sorbitol at a concentration of about 50 mM.

In some embodiments, the pharmaceutical composition comprises sorbitol at a concentration of about 100 mM.

In some embodiments, the pharmaceutical composition comprises sorbitol at a concentration of about 150 mM.

In some embodiments, the pharmaceutical composition comprises sorbitol at a concentration of about 200 mM.

In some embodiments, the pharmaceutical composition comprises sorbitol at a concentration of about 250 mM.

In some embodiments, the pharmaceutical composition comprises sorbitol at a concentration of about 300 mM.

In some embodiments, the pharmaceutical composition comprises sorbitol at a concentration of about 350 mM.

In some embodiments, the pharmaceutical composition comprises sorbitol at a concentration of about 400 mM.

In some embodiments, the pharmaceutical composition comprises sucrose at a concentration of from about 50 mM to about 500 mM.

In some embodiments, the pharmaceutical composition comprises sucrose at a concentration of from about 50 mM to about 450 mM.

In some embodiments, the pharmaceutical composition comprises sucrose at a concentration of from about 50 mM to about 400 mM.

In some embodiments, the pharmaceutical composition comprises sucrose at a concentration of from about 50 mM to about 350 mM.

In some embodiments, the pharmaceutical composition comprises sucrose at a concentration of from about 100 mM to about 350 mM.

In some embodiments, the pharmaceutical composition comprises sucrose at a concentration of from about 100 mM to about 200 mM.

In some embodiments, the pharmaceutical composition comprises sucrose at a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, the pharmaceutical composition comprises sucrose at a concentration of about 50 mM.

In some embodiments, the pharmaceutical composition comprises sucrose at a concentration of about 100 mM.

In some embodiments, the pharmaceutical composition comprises sucrose at a concentration of about 150 mM.

In some embodiments, the pharmaceutical composition comprises sucrose at a concentration of about 200 mM.

In some embodiments, the pharmaceutical composition comprises sucrose at a concentration of about 250 mM.

In some embodiments, the pharmaceutical composition comprises sucrose at a concentration of about 300 mM.

In some embodiments, the pharmaceutical composition comprises sucrose at a concentration of about 350 mM.

In some embodiments, the pharmaceutical composition comprises sucrose at a concentration of about 400 mM.

In some embodiments, the pharmaceutical composition comprises polysorbate.

In some embodiments, the pharmaceutical composition comprises polysorbate-20 (PS-20).

In some embodiments, the pharmaceutical composition comprises polysorbate-20 (PS-20) at a concentration of from about 0.01% (w/v) to about 0.1% (w/v).

In some embodiments, the pharmaceutical composition comprises polysorbate-20 (PS-20) at a concentration of from about 0.01% (w/v) to about 0.08% (w/v).

In some embodiments, the pharmaceutical composition comprises polysorbate-20 (PS-20) at a concentration of from about 0.01% (w/v) to about 0.04% (w/v).

In some embodiments, the pharmaceutical composition comprises polysorbate-20 (PS-20) at a concentration of from about 0.02% (w/v) to about 0.1% (w/v).

In some embodiments, the pharmaceutical composition comprises polysorbate-20 (PS-20) at a concentration of from about 0.02% (w/v) to about 0.08% (w/v).

In some embodiments, the pharmaceutical composition comprises polysorbate-20 (PS-20) at a concentration of from about 0.04% (w/v) to about 0.08% (w/v).

In some embodiments, the pharmaceutical composition comprises polysorbate-80 at a concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v) or about 0.1% (w/v).

In some embodiments, the pharmaceutical composition comprises polysorbate-80 at a concentration of from about 0.01% (w/v) to about 0.08% (w/v).

In some embodiments, the pharmaceutical composition comprises polysorbate-80 at a concentration of from about 0.01% (w/v) to about 0.04% (w/v).

In some embodiments, the pharmaceutical composition comprises polysorbate-80 at a concentration of from about 0.02% (w/v) to about 0.1% (w/v).

In some embodiments, the pharmaceutical composition comprises polysorbate-80 at a concentration of from about 0.02% (w/v) to about 0.08% (w/v).

In some embodiments, the pharmaceutical composition comprises polysorbate-80 at a concentration of from about 0.04% (w/v) to about 0.08% (w/v).

In some embodiments, the pharmaceutical composition comprises polysorbate-80 at a concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v) or about 0.1% (w/v).

In some embodiments, the pharmaceutical composition comprises polysorbate-20 and polysorbate 80.

In some embodiments, the pharmaceutical composition comprises polysorbate-20 and polysorbate-80 at a concentration of from about 0.01% (w/v) to about 0.08% (w/v).

In some embodiments, the pharmaceutical composition comprises polysorbate-20 and polysorbate-80 at a concentration of from about 0.01% (w/v) to about 0.04% (w/v).

In some embodiments, the pharmaceutical composition comprises polysorbate-20 and polysorbate-80 at a concentration of from about 0.02% (w/v) to about 0.1% (w/v).

In some embodiments, the pharmaceutical composition comprises polysorbate-20 and polysorbate-80 at a concentration of from about 0.02% (w/v) to about 0.08% (w/v).

In some embodiments, the pharmaceutical composition comprises polysorbate-20 and polysorbate-80 at a concentration of from about 0.04% (w/v) to about 0.08% (w/v).

In some embodiments, the pharmaceutical composition comprises polysorbate-20 and polysorbate-80 at a concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v) or about 0.1% (w/v).

In some embodiments, the pharmaceutical composition is at from about pH 5.0 to about 7.0.

In some embodiments, the pharmaceutical composition is at from about pH 5.0 to about 6.0.

In some embodiments, the pharmaceutical composition is at from about pH 5.3 to about 5.8.

In some embodiments, the pharmaceutical composition is at about pH 5.5.

In some embodiments, the pharmaceutical composition is at about pH 5.6.

In some embodiments, the pharmaceutically acceptable carrier comprises histidine, methionine, mannitol, sorbitol, polysorbate 20, polysorbate 80, or a combination thereof, and one or more salts (e.g., sodium chloride (NaCl), sodium acetate, etc.), wherein the pharmaceutical composition has a pH of 5 to 7.

In some embodiments, the pharmaceutical composition comprises:

about 1 mg/ml to about 180 mg/ml of the anti-CD38 antibody,
about 10 mM to about 50 mM sodium acetate,
about 10 mM to about 200 mM sodium chloride,
about 50 mM to about 250 mM D-mannitol,
about 0.01% to about 0.1% polysorbate 20,
about 0.01% to about 0.1% polysorbate 80,
about 2 mM to about 50 mM histidine,
about 2 mM to about 50 mM methionine, and
optionally, about 0.2 mg/ml to about 5.0 mg/mL rHuPH20 (about 75 kU/mL to about 150 kU/mL),
wherein the pharmaceutical composition has a pH of about 5.5 to about 6.5.

In some embodiments, the pharmaceutical composition comprises:
about 1 mg/ml to about 180 mg/ml of the anti-CD38 antibody,
about 1 mM to about 50 mM histidine,
about 50 mM to about 500 mM sorbitol,
about 0.1 mg/mL to about 5 mg/mL methionine, and
about 0.01% (w/v) to about 0.1% (w/v) polysorbate 20,
wherein the pharmaceutical composition has a pH of about 5.0 to about 6.5.

In some embodiments, the pharmaceutical composition comprises:
about 120 mg/mL of the anti-CD38 antibody,
about 10 mM histidine,
about 300 mM sorbitol,
about 1 mg/mL methionine, and
about 0.04% polysorbate 20,
wherein the pharmaceutical composition has a pH of about 5.6.

In some embodiments, the pharmaceutical composition comprises:
about 120 mg/mL of the anti-CD38 antibody,
about 10 mM histidine,
about 300 mM D-mannitol,
about 1 mg/mL methionine, and
about 0.04% Polysorbate 20,
wherein the pharmaceutical composition has a pH of about 5.6.

In some embodiments, the pharmaceutical composition comprises:
about 1 mg/mL to about 180 mg/mL of the anti-CD38 antibody,
about 5 mM to about 50 mM histidine,
about 50 mM to about 400 mM sorbitol, and
optionally, about 50 U/mL to about 5,000 U/mL of the hyaluronidase.

In some embodiments, the pharmaceutical composition comprises:
about 1 mg/mL to about 180 mg/mL of the anti-CD38 antibody,
about 5 mM to about 50 mM histidine,
about 50 mM to about 400 mM sorbitol,
about 0.01% (w/v) to about 0.1% PS-20,
about 0.1 mg/mL to about 2.5 mg/mL methionine, and
optionally, about 50 U/mL to about 5,000 U/mL hyaluronidase.

In some embodiments, the hyaluronidase is rHuPH20.

In some embodiments, the pharmaceutical composition comprises:
about 100 mg/mL to about 120 mg/mL of the anti-CD38 antibody,
about 10 mM histidine,
about 100 mM to about 300 mM sorbitol,
about 0.01% (w/v) to about 0.04% w/v PS-20,
about 1 mg/mL to about 2 mg/mL methionine, and
optionally, from about 50 U/mL to about 5,000 U/mL rHuPH20.

In some embodiments, the pharmaceutical composition comprises:
about 100 mg/mL of the anti-CD38 antibody,
about 10 mM histidine,
about 300 mM sorbitol,
about 0.04% (w/v) PS-20,
about 2 mg/mL methionine, and
optionally, about 500 U/mL rHuPH20,
wherein the pharmaceutical composition has a pH of about 5.5.

In some embodiments, the pharmaceutical composition comprises:
about 120 mg/mL of the anti-CD38 antibody,
about 10 mM histidine,
about 300 mM sorbitol,
about 0.04% (w/v) PS-20,
about 1 mg/mL methionine, and
optionally, about 2,000 U/mL rHuPH20,
wherein the pharmaceutical composition has a pH of about 5.6.

In some embodiments, the pharmaceutical composition comprises:
about 100 mg/mL of the anti-CD38 antibody,
about 10 mM histidine,
about 300 mM sorbitol,
about 2 mg/mL methionine, and
optionally, about 500 U/mL rHuPH20;
wherein the pharmaceutical composition has a pH of about 5.5.

In some embodiments, the pharmaceutical composition comprises:
about 100 mg/mL of the anti-CD38 antibody,
about 10 mM histidine,
about 300 mM sorbitol,
about 0.01% w/v PS-20,
about 2 mg/mL methionine, and
optionally, about 500 U/mL rHuPH20,
wherein the pharmaceutical composition has a pH of about 5.5.

In some embodiments, the pharmaceutical composition comprises:
about 100 mg/mL of the anti-CD38 antibody,
about 10 mM histidine,
about 300 mM sorbitol,
about 0.02% w/v PS-20,
about 2 mg/mL methionine, and
optionally, about 500 U/mL rHuPH20,
wherein the pharmaceutical composition has a pH of about 5.5.

The invention also provides a pharmaceutical composition comprising
about 100 mg/mL of the anti-CD38 antibody,
about 10 mM histidine,
about 300 mM sorbitol,
about 0.06% w/v PS-20,
about 2 mg/mL methionine, and
optionally, about 500 U/mL rHuPH20,
wherein the pharmaceutical composition has a pH of about 5.5.

In some embodiments, the pharmaceutical composition comprises:
about 100 mg/mL of the anti-CD38 antibody,
about 10 mM histidine,
about 300 mM sorbitol,
about 0.04% w/v PS-20,
about 1 mg/mL methionine, and
optionally, about 50 U/mL rHuPH20,
wherein the pharmaceutical composition has a pH of about 5.5.

In some embodiments, the pharmaceutical composition comprises:
about 100 mg/mL of the anti-CD38 antibody,
about 10 mM histidine,
about 300 mM sorbitol,
about 0.04% w/v PS-20,
about 1 mg/mL methionine, and
optionally, about 500 U/mL rHuPH20,
wherein the pharmaceutical composition has a pH of about 5.5.

In some embodiments, the pharmaceutical composition comprises:
about 100 mg/mL of the anti-CD38 antibody,
about 10 mM histidine,
about 300 mM sorbitol,
about 0.04% w/v PS-20,
about 1 mg/mL methionine, and
optionally, about 2,000 U/mL rHuPH20,
wherein the pharmaceutical composition has a pH of about 5.5.

In some embodiments, the pharmaceutical composition comprises:
about 100 mg/mL of the anti-CD38 antibody,
about 10 mM histidine,
about 300 mM sorbitol,
about 0.04% w/v PS-20,
about 1 mg/mL methionine, and
optionally, about 5,000 U/mL rHuPH20,
wherein the pharmaceutical composition has a pH of about 5.5.

Pharmaceutical compositions comprising the anti-CD38 antibody can be prepared by any method known in the art in view of the present disclosure. For example, the anti-CD38 antibody can be mixed with one or more pharmaceutically acceptable excipients to obtain a solution. The solution can be stored as a liquid at a temperature of about 2° C. to 8° C. and under protection from light exposure in an appropriate vial until administered to the subject.

In some embodiments, the pharmaceutical composition is prepared by mixing about 20 mg/ml of the anti-CD38 antibody with about 1.0 mg/mL rHuPH20 (75-150 kU/mL) prior to administration of the mixture to the subject, wherein the anti-CD38 antibody is in about 25 mM sodium acetate, about 60 mM sodium chloride, about 140 mM D-mannitol, about 0.04% polysorbate 20, at about pH 5.5, and rHuPH20 is in about 10 mM L-histidine, about 130 mM NaCl, about 10 mM L-methionine, and about 0.02% polysorbate 80, at about pH 6.5.

### Administration

The pharmaceutical compositions of the invention may be administered as a non-fixed combination.

The pharmaceutical compositions of the invention may also be administered as a fixed combination, e.g., as a unit dosage form (or dosage unit form). Fixed combinations may be advantageous for ease of administration and uniformity of dosage.

The invention also provides a unit dosage form, comprising the anti-CD38 antibody comprising the VH having sequence of SEQ ID NO: 4 and the VL having sequence of SEQ ID NO: 5 in an amount of from about 1,200 mg to about 5,000 mg and optionally, rHuPH20 in an amount of from about 30,000 U to about 75,000 U.

In some embodiments, the unit dosage form comprises
the anti-CD38 antibody in an amount of about 1,200 mg to about 4,000 mg, and
optionally, rHuPH20 in an amount of about 30,000 U to about 75,000 U.

In some embodiments, the unit dosage form comprises:
the anti-CD38 antibody in an amount of about 1,200 mg to about 2,400 mg, and
optionally, rHuPH20 in an amount of about 30,000 U to about 45,000 U.

In some embodiments, the unit dosage form comprises:
the anti-CD38 antibody in an amount of about 1,200 mg to about 1,800 mg, and
optionally, rHuPH20 in an amount of about 30,000 U to about 45,000 U.

In some embodiments, the unit dosage form comprises:
the anti-CD38 antibody in an amount of about 1,200 mg to about 5,000 mg,
histidine at a concentration of about 5 mM to about 15 mM,
sorbitol at a concentration of about 100 mM to about 300 mM,
PS-20 at a concentration of about 0.01% (w/v) to about 0.04% (w/v),
methionine at a concentration of about 1 mg/mL to about 2 mg/mL, and
optionally, rHuPH20 in an amount of about 30,000 U to about 75,000 U,
wherein the pharmaceutical composition has a pH of about 5.5.

In some embodiments, the unit dosage form comprises:
the anti-CD38 antibody in an amount of about 1,200 mg to about 2,400 mg,
histidine at a concentration of about 10 mM,
sorbitol at a concentration of about 300 mM,
PS-20 at a concentration of about 0.04% (w/v),
methionine at a concentration of from about 1 mg/mL, and
optionally, rHuPH20 in an amount of about 30,000 U to about 45,000 U,
wherein the pharmaceutical composition has a pH of about 5.5.

In some embodiments, the unit dosage form comprises:
the anti-CD38 antibody in an amount of about 1,200 mg to about 1,800 mg,
histidine at a concentration of about 10 mM,
sorbitol at a concentration of about 300 mM,
PS-20 at a concentration of about 0.04% (w/v),
methionine at a concentration of about 1 mg/mL, and
optionally, rHuPH20 in an amount of about 30,000 U to about 45,000 U,
wherein the pharmaceutical composition has a pH of about 5.5.

In some embodiments, the unit dosage form comprises:
the anti-CD38 antibody in an amount of about 1,200 mg to about 1,800 mg,
histidine at a concentration of about 5 mM to about 15 mM,
sorbitol at a concentration of about 100 mM to about 300 mM,
PS-20 at a concentration of about 0.01% (w/v) to about 0.04% (w/v),
methionine at a concentration of about 1 mg/mL to about 2 mg/mL, and
optionally, rHuPH20 in an amount of about 30,000 U to about 45,000 U,
wherein the pharmaceutical composition has a pH of about 5.5.

In some embodiments, the unit dosage form comprises:
the anti-CD38 antibody in an amount of about 1,800 mg,
histidine at a concentration of about 10 mM,
sorbitol at a concentration of about 300 mM,
PS-20 at a concentration of about 0.04% (w/v)
methionine at a concentration of about 1 mg/mL, and
optionally, rHuPH20 in an amount of about 30,000 U,
wherein the pharmaceutical composition has a pH of about 5.5.

In some embodiments, the unit dosage form comprises:
the anti-CD38 antibody in an amount of about 1,800 mg,
histidine at a concentration of about 10 mM,
sorbitol at a concentration of about 300 mM,
PS-20 at a concentration of about 0.04% (w/v),
methionine at a concentration of about 1 mg/mL, and
optionally, rHuPH20 in an amount of about 45,000 U,
wherein the pharmaceutical composition has a pH of about 5.5.

In some embodiments, the pharmaceutical composition is administered in a total volume of about 80 mL, about 90 mL, about 100 mL, about 110 mL or about 120 mL.

In some embodiments, the pharmaceutical composition is administered in a total volume of about 10 mL, about 11 mL, about 12 mL, about 13 mL, about 14 mL, about 15 mL, about 16 mL, about 17 mL, about 18 mL, about 19 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 80 mL, about 85 mL, about 90 mL, about 95 mL, about 100 mL, about 105 mL, about 110 mL, about 115 mL or about 120 mL.

In some embodiments, the pharmaceutical composition is administered in a total volume of about 10 mL.

In some embodiments, the pharmaceutical composition is administered in a total volume of about 15 mL.

In some embodiments, the pharmaceutical composition is administered in a total volume of about 20 mL.

The total volume of administration is typically smaller for the fixed combinations when compared to the non-fixed combinations.

In some embodiments, the unit dosage form of the pharmaceutical composition is stored in a container selected from a vial, a cartridge, a syringe, a prefilled syringe or a disposable pen.

In some embodiments, the total dosage of the anti-CD38 antibody can be administered to the subject in a single subcutaneous injection, or in multiple subcutaneous injections, such as 1, 2, 3, 4, 5, or more subcutaneous injections.

In some embodiments, the total dosage of the pharmaceutical composition is administered to the subject in a single subcutaneous injection per administration.

In some embodiments, the total dosage of the pharmaceutical composition is administered to the subject in multiple subcutaneous injections per administration, such as 2, 3, 4, or 5 subcutaneous injections.

In some embodiments, each subcutaneous injection lasts about 10 minutes to about 60 minutes.

In some embodiments, each subcutaneous injection lasts about 10 minutes to about 55 minutes.

In some embodiments, each subcutaneous injection lasts about 15 minutes to about 55 minutes.

In some embodiments, each subcutaneous injection lasts about 15 minutes to about 50 minutes.

In some embodiments, each subcutaneous injection lasts about 20 minutes to about 50 minutes.

In some embodiments, each subcutaneous injection lasts about 20 minutes to about 45 minutes.

In some embodiments, each subcutaneous injection lasts about 25 minutes to about 45 minutes.

In some embodiments, each subcutaneous injection lasts about 25 minutes to about 40 minutes.

In some embodiments, each subcutaneous injection lasts about 30 minutes to about 40 minutes.

In some embodiments, each subcutaneous injection lasts about 10 minutes, about 15 minutes, about 20 minutes, about 25 minutes, about 30 minutes, about 35 minutes, about 40 minutes, about 45 minutes, about 50 minutes, about 55 minutes, about 60 minutes or longer than 60 minutes.

In some embodiments, the total dosage of the pharmaceutical composition is administered once per day, once per week, once every 2 weeks, once per month, once every 2 months, once every 3 months, once every 4 months, once every 6 months, once every 9 months, for a period of one day, one week, 2 week, 3 week, one month, 2 months, 3 months, 4 months, 5 months, 6 months, 9 months, 1 year, 18 months, or 2 years or longer.

In some embodiments, the administration of the pharmaceutical composition is repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose. For example, the pharmaceutical compositions of the invention may be administered once weekly for eight weeks, followed by once in two weeks for 16 weeks, followed by once in four weeks.

In some embodiments, a total dosage of about 10 mg to about 600 mg of the anti-CD38 antibody is administered per administration (e.g., once per day for at least one day) by one subcutaneous injection or multiple subcutaneous injections (e.g., 2 to 5 injections).

In some embodiments, the dosage of the anti-CD38 antibody subcutaneous administered typically induces systemic injection related reactions.

In some embodiments, the dosage of the anti-CD38 antibody subcutaneous administered induced a systemic injection related reaction in the subject.

In some embodiments, a corticosteroid is administered to the subject prior to administration of the anti-CD38 antibody.

In some embodiments, premedication with the corticosteroid prevents a systemic injection related reaction and/or symptom thereof.

In some embodiments, premedication with the corticosteroid reduces a systemic injection related reaction and/or symptom thereof.

In some embodiments, the systemic injection related reaction has an immediate onset.

In some embodiments, the systemic injection related reaction has a delayed onset.

In some embodiments, the systemic injection related reaction and symptom thereof is selected from swelling and/or redness at the injection site, hypotension, shortness of breath, rash, uticaria, flushing, chest pain, fever, back pain, peripheral edema of extremities, vasovagal reactions, chills/rigor, nausea/emesis, headache, diaphoresis, lightheadedness, somnolence, or myalgia.

In some embodiments, the corticosteroid is administered prior to subcutaneous administration of the anti-CD38 antibody.

In some embodiments, the corticosteroid is administered just prior to subcutaneous administration of the anti-CD38 antibody.

In some embodiments, the corticosteroid is administered concomitantly with subcutaneous administration of the anti-CD38 antibody.

In some embodiments, the corticosteroid is administered about 1 minute to about 15 minutes prior to administration of the anti-CD38 antibody.

In some embodiments, the corticosteroid is administered about 5 minutes to about 15 minutes prior to administration of the anti-CD38 antibody.

In some embodiments, the corticosteroid is administered about 10 minutes to about 15 minutes prior to administration of the anti-CD38 antibody.

In some embodiments, the corticosteroid is administered about 0.5 hour to about 5 hours prior to administration of the anti-CD38 antibody.

In some embodiments, the corticosteroid is administered about 0.5 hour to about 4 hours prior to administration of the anti-CD38 antibody.

In some embodiments, the corticosteroid is administered about 1 hour to about 4 hours prior to administration of the anti-CD38 antibody.

In some embodiments, the corticosteroid is administered about 1 hour to about 2 hours prior to subcutaneous administration of the anti-CD38 antibody.

In some embodiments, the corticosteroid is administered about 0.5 hour, about 1 hour, about 1.5 hours, about 2 hours, about 2.5 hours, about 3 hours, about 3.5 hours, about 4 hours, about 4.5 hours or about 5 hours prior to administration of the anti-CD38 antibody.

The corticosteroid can be administered by any suitable method known in the art.

In some embodiments, the corticosteroid is administered orally.

In some embodiments, the corticosteroid is administered parenterally.

In some embodiments, the corticosteroid is selected from bethamethasone, prednisone, prednisolone, triamcinolone, methylprednisolone, dexamethasone, cortisol, hydrocortisone, or cortisone.

In some embodiments, the corticosteroid comprises or consists of prednisone.

In some embodiments, prednisone is administered orally.

In some embodiments, the corticosteroid is re-administered concomitantly with the administration of the anti-CD38 antibody.

In some embodiments, the corticosteroid is re-administered subsequent to (e.g., just after) the administration of the anti-CD38 antibody.

In some embodiments, the corticosteroid is re-administered about 1 minute to about 15 minutes subsequent to the administration of the anti-CD38 antibody.

In some embodiments, the corticosteroid is re-administered about 0.5 hour to about 10 hours subsequent to the administration of the anti-CD38 antibody.

In some embodiments, the corticosteroid is re-administered about 1 hour to about 10 hours subsequent to the administration of the anti-CD38 antibody.

In some embodiments, the corticosteroid is re-administered about 2 hours to about 10 hours subsequent to the administration of the anti-CD38 antibody.

In some embodiments, the corticosteroid is re-administered about 4 hours to about 10 hours subsequent to the administration of the anti-CD38 antibody.

In some embodiments, the corticosteroid is re-administered about 6 hours to about 10 hours subsequent to the administration of the anti-CD38 antibody.

In some embodiments, the corticosteroid is re-administered about 7 hours to about 9 hours subsequent to the administration of the anti-CD38 antibody.

In some embodiments, the corticosteroid is re-administered about 0.5 hour, about 1 hour, about 2 hours, about 3 hours, about 4 hours, about 5 hours, about 6 hours, about 7 hours, about 8 hours, about 9 hours, or about 10 hours subsequent to the administration of the anti-CD38 antibody.

In some embodiments, re-administration of the corticosteroid further prevents and/or reduces the risk of system injection related reactions associated with subcutaneous administration of the anti-CD38 antibody.

In some embodiments, the corticosteroid administered prior to administration of the anti-CD38 antibody and the corticosteroid re-administered subsequent to administration of the anti-CD38 antibody are the same.

In some embodiments, the corticosteroid administered prior to administration of the anti-CD38 antibody and the corticosteroid re-administered subsequent to administration of the anti-CD38 antibody are different.

In some embodiments, prednisone is orally administered about 1 hour to about 2 hours prior to subcutaneous administration of the anti-CD38 antibody, and is orally re-administered about 7 to 9 hours post-administration of the anti-CD38 antibody.

One of ordinary skill in the art would be able to determine the appropriate dosage of prednisone.

In some embodiments, about 10 mg to about 50 mg prednisone is administered prior to administration of the anti-CD38 antibody.

In some embodiments, about 15 mg to about 50 mg prednisone is administered prior to administration of the anti-CD38 antibody.

In some embodiments, about 15 mg to about 40 mg prednisone is administered prior to administration of the anti-CD38 antibody.

In some embodiments, about 20 mg to about 40 mg prednisone is administered prior to administration of the anti-CD38 antibody.

In some embodiments, about 20 mg to about 35 mg prednisone is administered prior to administration of the anti-CD38 antibody.

In some embodiments, about 25 mg to about 35 mg prednisone is administered prior to administration of the anti-CD38 antibody.

In some embodiments, about 20 mg to about 35 mg prednisone is administered prior to administration of the anti-CD38 antibody.

In some embodiments, about 10 mg, about 20 mg, about 30 mg, about 40 mg, or about 50 mg prednisone is administered prior to administration of the anti-CD38 antibody.

In some embodiments, about 10 mg to about 50 mg prednisone is administered subsequent to administration of the anti-CD38 antibody.

In some embodiments, about 15 mg to about 50 mg prednisone is administered subsequent to administration of the anti-CD38 antibody.

In some embodiments, about 15 mg to about 40 mg prednisone is administered subsequent to administration of the anti-CD38 antibody.

In some embodiments, about 20 mg to about 40 mg prednisone is administered subsequent to administration of the anti-CD38 antibody.

In some embodiments, about 20 mg to about 35 mg prednisone is administered subsequent to administration of the anti-CD38 antibody.

In some embodiments, about 25 mg to about 35 mg prednisone is administered subsequent to administration of the anti-CD38 antibody.

In some embodiments, about 20 mg to about 35 mg prednisone is administered subsequent to administration of the anti-CD38 antibody.

In some embodiments, about 10 mg, about 20 mg, about 30 mg, about 40 mg, or about 50 mg prednisone is administered subsequent to administration of the anti-CD38 antibody.

In some embodiments, the subject is administered an antihistamine, an antipyretic, or both prior to subcutaneous administration of the anti-CD38 antibody.

In some embodiments, the antihistamine and/or antipyretic is in addition to premedication with the corticosteroid.

In some embodiments, the antihistamine and/or antipyretic is the absence of premedication with the corticosteroid.

In some embodiments, the antihistamine comprises or consists of an H1 receptor antihistamine (e.g., dihphenhydramine).

In some embodiments, the antipyretic comprises or consists of acetaminophen.

In some embodiments, the antihistamine and/or antipyretic is administered about 1 hour before subcutaneous administration of the anti-CD38 antibody.

### Monitoring

According to embodiments of the invention, a variety of factors can be analyzed to determine whether a particular dosage of an anti-CD38 antibody provides for safe subcutaneous administration. For example, safety of a certain dosage of subcutaneously administered anti-CD38 antibody can be assessed by immunogenicity studies (e.g., measuring the production of anti-daratumumab antibodies); evaluating changes in CD38 expression levels; assessing the degree and duration of depletion of CD38 expressing cell counts (e.g., plasma cells, natural killer (NK) cells, percent total of lymphocytes); and determining the effects on blood biomarkers, such as serum proteins (e.g., cytokines, chemokines, and inflammatory proteins) by protein profiling. The safety of subcutaneously administered anti-CD38 antibody can also be monitored by physical examination of the subject; observation of local injection site reactions, systemic injection related reactions and other allergic reactions; electrocardiograms; clinical laboratory tests; vital signs; concomitant medications; and monitoring of other adverse events.

In some embodiments, the method further comprises measuring a production of antibodies specific for the anti-CD38 antibody in the subject after subcutaneous administration of the anti-CD38 antibody.

In some embodiments, the method further comprises measuring a change in CD38 expression level in the subject after subcutaneous administration of the anti-CD38 antibody.

In some embodiments, the method further comprises measuring a degree of depletion of CD38 expressing cells in the subject after subcutaneous administration of the anti-CD38 antibody.

In some embodiments, the method further comprises measuring a duration of depletion of CD38 expressing cells in the subject after subcutaneous administration of the anti-CD38 antibody.

In some embodiments, the CD38 expressing cells comprise plasma cells, NK cells, lymphocytes, or a combination thereof.

In some embodiments, the method further comprises profiling biomarkers in the subject after subcutaneous administration of the anti-CD38 antibody.

In some embodiments, the biomarkers comprise blood biomarkers.

In some embodiments, the biomarkers comprise serum proteins (e.g., cytokines, chemokines, and inflammatory proteins).

In some embodiments, the method further comprises physically examining the subject after subcutaneous administration of the anti-CD38 antibody.

In some embodiments, the method further comprises detecting an allergic reaction (e.g., a local injection site reaction or a systemic injection related reaction) in the subject after subcutaneous administration of the anti-CD38 antibody.

In some embodiments, the method further comprises performing an electrocardiogram in the subject after subcutaneous administration of the anti-CD38 antibody.

NK cells are a type of cytotoxic lymphocyte important for the innate immune system, are one of the key effector cells for ADCC-mediated depletion of CD38⁺ cells. NK cells are known to express CD38, thus the number of NK cells in circulation may decline following anti-CD38 antibody treatment. Additionally, plasma cells express CD38 and thus will be susceptible to the anti-CD38 antibody mediated cell lysis. Plasma cells are white blood cells that secrete antibody molecules, which recognize and bind foreign substances, and initiate neutralization or destruction of the substance. Depletion of NK cells and plasma cells is measured relative to the amount of NK cells and plasma cells in the subject prior to administration of the anti-CD38 antibody. Any method known in the art in view of the present disclosure can be used to determine the depletion of NK cells and plasma cells, including, but not limited to, flow cytometry.

In some embodiments, the subject has less than about 80% depletion of NK cells about four (4) weeks after subcutaneous administration of the anti-CD38 antibody.

In some embodiments, the subject has less than about 70% depletion of NK cells about four (4) weeks after subcutaneous administration of the anti-CD38 antibody.

In some embodiments, the subject has less than about 60% depletion of NK cells about four (4) weeks after subcutaneous administration of the anti-CD38 antibody.

In some embodiments, the subject has less than about 50% depletion of NK cells about four (4) weeks after subcutaneous administration of the anti-CD38 antibody.

In some embodiments, the subject has less than about 40% depletion of NK cells about four (4) weeks after subcutaneous administration of the anti-CD38 antibody.

In some embodiments, the subject has less than about 30% depletion of NK cells about four (4) weeks after subcutaneous administration of the anti-CD38 antibody.

In some embodiments, the subject has less than about 20% depletion of NK cells about four (4) weeks after subcutaneous administration of the anti-CD38 antibody.

In some embodiments, the subject has less than about 10% depletion of NK cells about two (2) weeks after subcutaneous administration of the anti-CD38 antibody.

In some embodiments, the subject has less than about 80% depletion of NK cells about two (2) weeks after subcutaneous administration of the anti-CD38 antibody.

In some embodiments, the subject has less than about 70% depletion of NK cells about two (2) weeks after subcutaneous administration of the anti-CD38 antibody.

In some embodiments, the subject has less than about 60% depletion of NK cells about two (2) weeks after subcutaneous administration of the anti-CD38 antibody.

In some embodiments, the subject has less than about 50% depletion of NK cells about two (2) weeks after subcutaneous administration of the anti-CD38 antibody.

In some embodiments, the subject has less than about 40% depletion of NK cells about two (2) weeks after subcutaneous administration of the anti-CD38 antibody.

In some embodiments, the subject has less than about 30% depletion of NK cells about two (2) weeks after subcutaneous administration of the anti-CD38 antibody.

In some embodiments, the subject has less than about 20% depletion of NK cells about two (2) weeks after subcutaneous administration of the anti-CD38 antibody.

In some embodiments, the subject has less than about 10% depletion of NK cells about two (2) weeks after subcutaneous administration of the anti-CD38 antibody.

In some embodiments, the subject has less than about 80% depletion of plasma cells about four (4) weeks after subcutaneous administration of the anti-CD38 antibody.

In some embodiments, the subject has less than about 70% depletion of plasma cells about four (4) weeks after subcutaneous administration of the anti-CD38 antibody.

In some embodiments, the subject has less than about 60% depletion of plasma cells about four (4) weeks after subcutaneous administration of the anti-CD38 antibody.

In some embodiments, the subject has less than about 50% depletion of plasma cells about four (4) weeks after subcutaneous administration of the anti-CD38 antibody.

In some embodiments, the subject has less than about 40% depletion of plasma cells about four (4) weeks after subcutaneous administration of the anti-CD38 antibody.

In some embodiments, the subject has less than about 30% depletion of plasma cells about four (4) weeks after subcutaneous administration of the anti-CD38 antibody.

In some embodiments, the subject has less than about 20% depletion of plasma cells about four (4) weeks after subcutaneous administration of the anti-CD38 antibody.

In some embodiments, the subject has less than about 10% depletion of plasma cells about two (2) weeks after subcutaneous administration of the anti-CD38 antibody.

In some embodiments, the subject has less than about 80% depletion of plasma cells about two (2) weeks after subcutaneous administration of the anti-CD38 antibody.

In some embodiments, the subject has less than about 70% depletion of plasma cells about two (2) weeks after subcutaneous administration of the anti-CD38 antibody.

In some embodiments, the subject has less than about 60% depletion of plasma cells about two (2) weeks after subcutaneous administration of the anti-CD38 antibody.

In some embodiments, the subject has less than about 50% depletion of plasma cells about two (2) weeks after subcutaneous administration of the anti-CD38 antibody.

In some embodiments, the subject has less than about 40% depletion of plasma cells about two (2) weeks after subcutaneous administration of the anti-CD38 antibody.

In some embodiments, the subject has less than about 30% depletion of plasma cells about two (2) weeks after subcutaneous administration of the anti-CD38 antibody.

In some embodiments, the subject has less than about 20% depletion of plasma cells about two (2) weeks after subcutaneous administration of the anti-CD38 antibody.

In some embodiments, the subject has less than about 10% depletion of plasma cells about two (2) weeks after subcutaneous administration of the anti-CD38 antibody.

In a preferred embodiment, subcutaneous administration of the anti-CD38 antibody results in 50% or less depletion of NK cells or plasma cells for at least two (2) weeks after administration of the anti-CD38 antibody.

In another general aspect, the invention relates to a method of providing subcutaneous administration (e.g., providing safe subcutaneous administration) of an anti-CD38 antibody to a subject in need thereof, the method comprising subcutaneously administering to the subject a pharmaceutical composition comprising the anti-CD38 antibody and a pharmaceutically acceptable carrier, wherein the anti-CD38 antibody comprises HCDR1, HCDR2 and HCDR3 amino acid sequences of SEQ ID NOs: 6, 7 and 8, respectively, and LCDR1, LCDR2 and LCDR3 amino acid sequences of SEQ ID NOs: 9, 10 and 11, respectively, and wherein the anti-CD38 antibody is subcutaneously administered with rHuPH20.

In another general aspect, the invention relates to a method of providing subcutaneous administration (e.g., providing safe subcutaneous administration) of an anti-CD38 antibody to a subject in need thereof, the method comprising subcutaneously administering to the subject a pharmaceutical composition comprising the anti-CD38 antibody and a pharmaceutically acceptable carrier, wherein the anti-CD38 antibody comprises HCDR1, HCDR2 and HCDR3 amino acid sequences of SEQ ID NOs: 6, 7 and 8, respectively, and LCDR1, LCDR2 and LCDR3 amino acid sequences of SEQ ID NOs: 9, 10 and 11, respectively, and wherein the anti-CD38 antibody is subcutaneously administered without recombinant human hyaluronidase.

In yet another general aspect, the invention relates to a method of providing treatment of an autoimmune disease to a subject in need thereof, the method comprising subcutaneously administering to the subject a pharmaceutical composition comprising an anti-CD38 antibody and a pharmaceutically acceptable carrier, wherein the anti-CD38 antibody comprises HCDR1, HCDR2 and HCDR3 amino acid sequences of SEQ ID NOs: 6, 7 and 8, respectively, and LCDR1, LCDR2 and LCDR3 amino acid sequences of SEQ ID NOs: 9, 10 and 11, respectively, and wherein a total dosage of the anti-CD38 antibody administered is about 10 mg to about 600 mg per administration. Any of the methods described herein for safe administration of the anti-CD38 antibody can be used to provide safe treatment of an autoimmune disease in a subject in need thereof.

In some embodiments of the invention, treatment with the anti-CD38 antibody results in less than 80% depletion of NK cells or plasma cells for at least four (4) weeks after administration of daratumumab.

In certain embodiments, a corticosteroid is administered preferably orally, to the subject, prior to administration of the anti-CD38 antibody, and is optionally re-administered subsequent to administration of the anti-CD38 antibody. In a preferred embodiment, the corticosteroid is prednisone.

In other embodiments, the anti-CD38 antibody is administered without recombinant human hyaluronidase.

In one non-limiting regimen of providing subcutaneous administration (e.g., providing safe subcutaneous administration) of an anti-CD38 antibody and/or treatment (e.g., safe treatment) of an autoimmune disease, a subject is subcutaneously administered a pharmaceutical composition comprising about 120 mg/mL of the anti-CD38 antibody. The total volume of the composition administered is appropriately adjusted to provide the target dosage, i.e., about 10 mg to about 600 mg, in a single subcutaneous injection, or in multiple subcutaneous injections, preferably in a single subcutaneous injection.

In another non-limiting regimen of providing subcutaneous administration (e.g., providing safe subcutaneous administration) of an anti-CD38 antibody and/or treatment (e.g., safe treatment) of an autoimmune disease, a subject is orally administered about 40 mg of prednisone. About 1 to 2 hours subsequent to prednisone administration, the subject is subcutaneously administered a pharmaceutical composition comprising about 120 mg/mL of the anti-CD38 antibody. The total volume of the composition administered is appropriately adjusted to provide the target dosage, i.e., about 10 mg to about 600 mg, in a single subcutaneous injection. Optionally, about 7 to 9 hours subsequent to administration of daratumumab, about 20 mg of prednisone is orally re-administered to the subject.

In some embodiments, “safe treatment” and “safe administration” when used with respect to subcutaneous administration of daratumumab, mean reduced adverse events including, but not limited to, reduced depletion of CD38⁺ cells, such as plasma cells, NK cells, T-cells, B-cells, etc., particularly NK cells and/or plasma cells. In a particular embodiment, “safe treatment” and “safe administration” mean that subcutaneous administration of an anti-CD38 antibody (such as daratumumab) results in less than 80% depletion of CD38⁺ cells (e.g., plasma cells, NK cells, T-cells, B-cells, etc.), preferably for at least four (4) weeks after administration of daratumumab. NK cells are a type of lymphocyte (white blood cell) and a component of the innate immune system. NK cells are cytotoxic, and play a role in, e.g., host-rejection of tumors and virally infected cells.

### Embodiments

Embodiment 1 is a method of providing subcutaneous administration of an anti-CD38 antibody to a subject in need thereof, the method comprising subcutaneously administering to the subject a pharmaceutical composition comprising the anti-CD38 antibody and a pharmaceutically acceptable carrier, wherein a total dosage of the anti-CD38 antibody is about 10 mg to about 2,400 mg per administration.

Embodiment 2 is a method of providing treatment of an autoimmune disease in a subject in need thereof, the method comprising subcutaneously administering to the subject a pharmaceutical composition comprising an anti-CD38 antibody and a pharmaceutically acceptable carrier, and wherein a total dosage of the anti-CD38 antibody administered is about 10 mg to about 2,400 mg per administration.

Embodiment 3 is the method of embodiment 1 or 2, wherein the total dosage of the anti-CD38 antibody is about 10 mg to about 600 mg per administration.

Embodiment 4 is the method of embodiment 3, wherein the total dosage of the anti-CD38 antibody is about 10 mg, about 20 mg, about 25 mg, about 30 mg, about 40 mg, about 50 mg, about 60 mg, about 70 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg or about 600 mg per administration.

Embodiment 5 is the method of any one of embodiments 1-4, wherein the total dosage of the anti-CD38 antibody is administered in a single subcutaneous injection.

Embodiment 6 is the method of any one of embodiments 1-4, wherein the total dosage of the anti-CD38 antibody is administered in multiple subcutaneous injections, e.g., two to five injections.

Embodiment 7 is the method of any one of embodiments 1-6, wherein a corticosteroid is administered to the subject prior to the administration of the anti-CD38 antibody, and is optionally re-administered subsequent to the administration of the anti-CD38 antibody.

Embodiment 8 is the method of any one of embodiments 1-6, wherein a corticosteroid is administered to the subject subsequent to the administration of the anti-CD38 antibody.

Embodiment 9 is the method of embodiment 7 or 8, wherein the corticosteroid is prednisone.

Embodiment 10 is the method of any one of embodiments 7-9, wherein the corticosteroid is administered orally.

Embodiment 11 is the method of any one of embodiments 1-10, wherein the administration of the anti-CD38 antibody results in less than 80% depletion of natural killer (NK) cells or plasma cells for at least four (4) weeks after administration of the anti-CD38 antibody.

Embodiment 12 is the method of any one of embodiments 1-10, wherein the administration of the anti-CD38 antibody results in less than 80% depletion of NK cells or plasma cells four (4) weeks after administration of the anti-CD38 antibody.

Embodiment 13 is the method of any one of embodiments 1-10, wherein the administration of the anti-CD38 antibody results in the subject having greater than 80% depletion of NK cells or plasma cells for no more than four (4) weeks.

Embodiment 14 is the method of any one of embodiments 1-13, wherein the anti-CD38 antibody is subcutaneously administered without recombinant human hyaluronidase.

Embodiment 15 is the method of any one of embodiments 1-14, wherein the pharmaceutical composition is a solution.

Embodiment 16 is the method of embodiment 15, wherein the solution comprises the anti-CD38 antibody at a concentration of about 1 mg/mL to about 180 mg/mL.

Embodiment 17 is the method of embodiment 16, wherein the concentration of the anti-CD38 antibody is about 1 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, or about 180 mg/mL.

Embodiment 18 is the method of any one of embodiments 1-16, wherein the pharmaceutical composition comprises: about 1 mg/ml to about 180 mg/ml of the anti-CD38 antibody, about 1 mM to about 50 mM histidine, about 50 mM to about 500 mM sorbitol, about 0.1 mg/mL to about 5 mg/mL methionine, and about 0.01% (w/v) to about 0.1% (w/v) polysorbate 20, at a pH 5.0-6.5.

Embodiment 19 is the method of any one of embodiments 1-16, wherein the pharmaceutical composition comprises: about 120 mg/mL of the anti-CD38 antibody, about 10 mM histidine, about 300 mM sorbitol, about 1 mg/mL methionine, and about 0.04% polysorbate 20, preferably at about pH 5.6.

Embodiment 20 is the method of any one of embodiments 1-16, wherein the pharmaceutical composition comprises: about 120 mg/mL of the anti-CD38 antibody, about 10 mM histidine, about 300 mM D-mannitol, about 1 mg/mL methionine, and about 0.04% Polysorbate 20, at about pH 5.6.

Embodiment 21 is the method of any one of embodiments 1-17, wherein the subject in need thereof has or is suspected of having a disease selected from the group consisting of lupus, systemic lupus erythematosus, Sjogren's Syndrome, arthritis, rheumatoid arthritis, asthma, COPD, pelvic inflammatory disease, Alzheimer's Disease, inflammatory bowel disease, Crohn's disease, ulcerative colitis, Peyronie's Disease, celiac disease, gallbladder disease, Pilonidal disease, peritonitis, psoriasis, psoriatic arthritis, vasculitis, surgical adhesions, stroke, Type I Diabetes, Lyme disease, meningoencephalitis, autoimmune uveitis, multiple sclerosis, Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease, transplant rejection, heart disease including ischaemic diseases such as myocardial infarction as well as atherosclerosis, intravascular coagulation, bone resorption, osteoporosis, osteoarthritis, periodontitis and hypochlorhydia, infertility related to lack of fetal-maternal tolerance, vitiligo, myasthenia gravis or systemic sclerosis.

Embodiment 22 is the method of any one of embodiments 1-20, wherein the subject is diagnosed with or suspected of having an autoimmune disease.

Embodiment 23 is the method of embodiment 22, wherein the autoimmune disease is selected from the group consisting of arthritis, rheumatoid arthritis (RA), psoriatic arthritis, ankylosing spondylitis, ulcerative colitis, plaque psoriasis, systemic lupus erythematosus (SLE), inflammatory bowel disease and Crohn's disease.

Embodiment 24 is the method of embodiment 23, wherein the autoimmune disease is RA.

Embodiment 25 is the method of embodiment 23, wherein the autoimmune disease is SLE.

Embodiment 26 is the method of any one of embodiments 1-25, wherein the anti-CD38 antibody comprises heavy chain complementarity determining region 1 (HCDR1), HCDR2 and HCDR3 amino acid sequences of SEQ ID NOs: 6, 7 and 8, respectively, and light chain complementarity determining region 1 (LCDR1), LCDR2 and LCDR3 amino acid sequences of SEQ ID NOs: 9, 10 and 11, respectively.

Embodiment 27 is the method of any one of embodiments 1-26, wherein the anti-CD38 antibody comprises a heavy chain variable region sequence of SEQ ID NO: 4 and a light chain variable region sequence of SEQ ID NO: 5.

Embodiment 28 is the method of any one of embodiments 1-27, wherein the anti-CD38 antibody comprises a heavy chain sequence of SEQ ID NO: 12 and a light chain sequence of SEQ ID NO: 13.

Embodiment 29 is the method of any one of embodiments 1-28, wherein the anti-CD38 antibody is of an IgG1 isotype.

Embodiment 30 is the method of any one of embodiments 1-29, wherein the subject is a human.

Embodiment 31 is a method of preparing a pharmaceutical composition comprising an anti-CD38 antibody for subcutaneous administration to a subject in need thereof, the method comprising combining about 10 mg to about 2,400 mg of the anti-CD38 antibody with at least one pharmaceutically acceptable carrier.

Embodiment 32 is the method of embodiment 31, wherein the method comprising combining about 10 mg to about 600 mg of the anti-CD38 antibody with at least one pharmaceutically acceptable carrier.

Embodiment 33 is a pharmaceutical composition for use in providing subcutaneous administration of an anti-CD38 antibody to a subject in need thereof or providing treatment of an autoimmune disease to a subject in need thereof, the pharmaceutical composition comprising the anti-CD38 antibody and a pharmaceutically acceptable carrier, wherein a total dosage of the anti-CD38 antibody administered is about 10 mg to about 2,400 mg per administration, and the pharmaceutical composition is formulated for subcutaneous administration.

Embodiment 34 is the pharmaceutical composition for use of embodiment 33, wherein the total dosage of the anti-CD38 antibody administered is about 10 mg to about 600 mg per administration.

Embodiment 35 is the pharmaceutical composition for use of embodiment 33 or 34, wherein the total dosage of the anti-CD38 antibody is administered in a single subcutaneous injection.

Embodiment 36 is the pharmaceutical composition for use of embodiments 33-35, wherein the total dosage of the anti-CD38 antibody is administered in multiple subcutaneous injections, e.g., two to five injections.

Embodiment 37 is the pharmaceutical composition for use of any one of embodiments 33-36, wherein a corticosteroid is administered to the subject prior to the administration of the anti-CD38 antibody, and is optionally re-administered subsequent to the administration of the anti-CD38 antibody.

Embodiment 38 is the pharmaceutical composition for use of any one of embodiments 33-36, wherein the corticosteroid is administered subsequent to the administration of the anti-CD38 antibody.

Embodiment 39 is the pharmaceutical composition for use of embodiment 37 or 38, wherein the corticosteroid is prednisone.

Embodiment 40 is the pharmaceutical composition for use of any one of embodiments 37-39, wherein the corticosteroid is administered orally.

Embodiment 41 is the pharmaceutical composition for use of any one of embodiments 33-40, wherein the administration of the anti-CD38 antibody results in less than 80% depletion of NK cells or plasma cells for at least four (4) weeks after administration of the anti-CD38 antibody.

Embodiment 42 is the pharmaceutical composition for use of any one of embodiments 33-40, wherein the administration of the anti-CD38 antibody results in less than 80% depletion of NK cells or plasma cells four (4) weeks after administration of the anti-CD38 antibody.

Embodiment 43 is the pharmaceutical composition for use of any one of embodiments 33-40, wherein the administration of the anti-CD38 antibody results in the subject having greater than 80% depletion of NK cells or plasma cells for no more than four (4) weeks.

Embodiment 44 is the pharmaceutical composition for use of any one of embodiments 33-43, wherein the anti-CD38 antibody is subcutaneously administered without recombinant human hyaluronidase.

Embodiment 45 is the pharmaceutical composition for use of any one of embodiments 33-44, wherein the total dosage of the anti-CD38 antibody is about 10 mg, about 20 mg, about 25 mg, about 30 mg, about 40 mg, about 50 mg, about 60 mg, about 70 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg or about 600 mg.

Embodiment 46 is the pharmaceutical composition for use of any one of embodiments 33-45, wherein the pharmaceutical composition is a solution.

Embodiment 47 is the pharmaceutical composition for use of embodiment 46, wherein the solution comprises the anti-CD38 antibody at a concentration of about 1 mg/mL to about 180 mg/mL.

Embodiment 48 is the pharmaceutical composition for use of embodiment 47, wherein the concentration of the anti-CD38 antibody is about 1 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, or about 180 mg/mL.

Embodiment 49 is the pharmaceutical composition for use of any one of embodiments 33-48, wherein the pharmaceutical composition comprises: about 1 mg/ml to about 180 mg/ml of the anti-CD38 antibody, about 1 mM to about 50 mM histidine, about 50 mM to about 500 mM sorbitol, about 0.1 mg/mL to about 5 mg/mL methionine, and about 0.01% (w/v) to about 0.1% (w/v) polysorbate 20, at a pH 5.0-6.5.

Embodiment 50 is the pharmaceutical composition for use of any one of embodiments 33-48, wherein the pharmaceutical composition comprises: about 120 mg/mL of the anti-CD38 antibody, about 10 mM histidine, about 300 mM sorbitol, about 1 mg/mL methionine, and about 0.04% polysorbate 20, at about pH 5.6.

Embodiment 51 is the pharmaceutical composition for use of any one of embodiments 33-48, wherein the pharmaceutical composition comprises: about 120 mg/mL of the anti-CD38 antibody, about 10 mM histidine, about 300 mM D-mannitol, about 1 mg/mL methionine, and about 0.04% Polysorbate 20, at about pH 5.6.

Embodiment 52 is pharmaceutical composition for use of any one of embodiments 33-51, wherein the subject in need thereof has or is suspected of having a disease selected from the group consisting of lupus, systemic lupus erythematosus, Sjogren's Syndrome, arthritis, rheumatoid arthritis, asthma, COPD, pelvic inflammatory disease, Alzheimer's Disease, inflammatory bowel disease, Crohn's disease, ulcerative colitis, Peyronie's Disease, celiac disease, gallbladder disease, Pilonidal disease, peritonitis, psoriasis, psoriatic arthritis, vasculitis, surgical adhesions, stroke, Type I Diabetes, Lyme disease, meningoencephalitis, autoimmune uveitis, multiple sclerosis, Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease, transplant rejection, heart disease including ischaemic diseases such as myocardial infarction as well as atherosclerosis, intravascular coagulation, bone resorption, osteoporosis, osteoarthritis, periodontitis and hypochlorhydia, infertility related to lack of fetal-maternal tolerance, vitiligo, myasthenia gravis or systemic sclerosis.

Embodiment 53 is the pharmaceutical composition for use of any one of embodiments 33-51, wherein the autoimmune disease is selected from the group consisting of arthritis, RA, psoriatic arthritis, ankylosing spondylitis, ulcerative colitis, plaque psoriasis, SLE, lupus nephritis, ANCA associated vasculitis, myasthenia gravis, progressive multiple sclerosis, IgG4 related diseases, Sjogren's syndrome, immune thrombocytopenic purpura, transplant rejection, inflammatory bowel disease and Crohn's disease.

Embodiment 54 is the pharmaceutical composition for use of embodiment 53, wherein the autoimmune diseases is RA.

Embodiment 55 is the pharmaceutical composition for use of embodiment 53, wherein the autoimmune diseases is SLE.

Embodiment 56 is the pharmaceutical composition for use of any one of embodiments 33-55, wherein the anti-CD38 antibody comprises HCDR1, HCDR2 and HCDR3 amino acid sequences of SEQ ID NOs: 6, 7 and 8, respectively, and LCDR1, LCDR2 and LCDR3 amino acid sequences of SEQ ID NOs: 9, 10 and 11, respectively.

Embodiment 57 is the pharmaceutical composition for use of any one of embodiments 33-56, wherein the anti-CD38 antibody comprises a heavy chain variable region sequence of SEQ ID NO: 4 and a light chain variable region sequence of SEQ ID NO: 5.

Embodiment 58 is the pharmaceutical composition for use of any one of embodiments 33-57, wherein the anti-CD38 antibody comprises a heavy chain sequence of SEQ ID NO: 12 and a light chain sequence of SEQ ID NO: 13.

Embodiment 59 is the pharmaceutical composition for use of any one of embodiments 33-58, wherein the anti-CD38 antibody is of an IgG1 isotype.

Embodiment 60 is the pharmaceutical composition for use of any one of embodiments 33-59, wherein the subject is a human.

Embodiment 61 is a use of an anti-CD38 antibody in the manufacture of a medicament for providing subcutaneous administration of the anti-CD38 antibody to a subject in need thereof or providing treatment of an autoimmune disease to a subject in need thereof, wherein a total dosage of the anti-CD38 antibody administered is about 10 mg to about 2,400 mg per administration.

Embodiment 62 is the use of embodiment 61, wherein the total dosage of the anti-CD38 antibody administered is about 10 mg to about 600 mg per administration.

Embodiment 63 is the use of embodiment 61 or 62, wherein the total dosage of the anti-CD38 antibody is administered in a single subcutaneous injection.

Embodiment 64 is the use of embodiment 61 or 62, wherein the total dosage of the anti-CD38 antibody is administered in multiple subcutaneous injections, e.g., two to five injections.

Embodiment 65 is the use of any one of embodiments 61-64, wherein a corticosteroid is administered to the subject prior to the administration of the anti-CD38 antibody, and is optionally re-administered subsequent to the administration of the anti-CD38 antibody.

Embodiment 66 is the use of any one of embodiments 61-64, wherein the corticosteroid is administered subsequent to the administration of the anti-CD38 antibody.

Embodiment 67 is the use of embodiment 65 or 66, wherein the corticosteroid is prednisone.

Embodiment 68 is the use of any one of embodiments 65-67, wherein the corticosteroid is administered orally.

Embodiment 69 is the use of any one of embodiments 61-68, wherein the administration of the anti-CD38 antibody results in less than 80% depletion of NK cells or plasma cells for at least four (4) weeks after administration of the anti-CD38 antibody.

Embodiment 70 is the use of any one of embodiments 61-68, wherein the administration of the anti-CD38 antibody results in less than 80% depletion of NK cells or plasma cells four (4) weeks after administration of the anti-CD38 antibody.

Embodiment 71 is the use of any one of embodiments 61-68, wherein the administration of the anti-CD38 antibody results in the subject having greater than 80% depletion of NK cells or plasma cells for no more than four (4) weeks.

Embodiment 72 is the use of any one of embodiments 61-71, wherein the anti-CD38 antibody is subcutaneously administered without recombinant human hyaluronidase.

Embodiment 73 is the use of any one of embodiments 61-72, wherein the total dosage of the anti-CD38 antibody is about 10 mg, about 20 mg, about 25 mg, about 30 mg, about 40 mg, about 50 mg, about 60 mg, about 70 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg or about 600 mg.

Embodiment 74 is the use of any one of embodiments 61-73, wherein the anti-CD38 antibody is manufactured as a solution.

Embodiment 75 is the use of embodiment 74, wherein the solution comprises the anti-CD38 antibody at a concentration of about 1 mg/mL to about 180 mg/mL.

Embodiment 76 is the use of embodiment 75, wherein the concentration of the anti-CD38 antibody is about 1 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, or about 180 mg/mL.

Embodiment 77 is the use of any one of embodiments 61-75, wherein the medicament comprises: about 1 mg/ml to about 180 mg/ml of the anti-CD38 antibody, about 1 mM to 50 mM histidine, about 50 mM to about 500 mM sorbitol, about 0.1 mg/mL to about 5 mg/mL methionine, and about 0.01% (w/v) to about 0.1% (w/v) polysorbate 20, at a pH 5.0-6.5.

Embodiment 78 is the use of any one of embodiments 61-76, wherein the medicament comprises: about 120 mg/mL of the anti-CD38 antibody, about 10 mM histidine, about 300 mM sorbitol, about 1 mg/mL methionine, and about 0.04% polysorbate 20, at about pH 5.6.

Embodiment 79 is the use of any one of embodiments 61-76, wherein the medicament comprises: about 120 mg/mL of the anti-CD38 antibody, about 10 mM histidine, about 300 mM D-mannitol, about 1 mg/mL methionine, and about 0.04% Polysorbate 20, at about pH 5.6.

Embodiment 80 is the use of any one of embodiments 61-76, wherein the autoimmune disease is selected from the group consisting of arthritis, RA, psoriatic arthritis, ankylosing spondylitis, ulcerative colitis, plaque psoriasis, SLE, inflammatory bowel disease and Crohn's disease.

Embodiment 81 is the use of embodiment 80, wherein the autoimmune diseases is RA.

Embodiment 82 is the use of embodiment 80, wherein the autoimmune diseases is SLE.

Embodiment 83 is the use of any one of embodiments 61-82, wherein the anti-CD38 antibody comprises HCDR1, HCDR2 and HCDR3 amino acid sequences of SEQ ID NOs: 6, 7 and 8, respectively, and LCDR1, LCDR2 and LCDR3 amino acid sequences of SEQ ID NOs: 9, 10 and 11, respectively.

Embodiment 84 is the use of any one of embodiments 61-83, wherein the anti-CD38 antibody comprises a heavy chain variable region sequence of SEQ ID NO: 4 and a light chain variable region sequence of SEQ ID NO: 5.

Embodiment 85 is the use of any one of embodiments 61-84, wherein the anti-CD38 antibody comprises a heavy chain sequence of SEQ ID NO: 12 and a light chain sequence of SEQ ID NO: 13.

Embodiment 86 is the use of any one of embodiments 61-85, wherein the anti-CD38 antibody is of an IgG1 isotype.

Embodiment 87 is the use of any one of embodiments 61-86, wherein the subject is a human.

Embodiment 88 is a pharmaceutical composition comprising: about 1 mg/ml to about 180 mg/ml of an anti-CD38 antibody, about 1 mM to about 50 mM histidine, about 50 mM to about 500 mM sorbitol, about 0.1 mg/mL to about 5 mg/mL methionine, and about 0.01% (w/v) to about 0.1% (w/v) polysorbate 20, at a pH 5.0-6.5.

Embodiment 89 is a pharmaceutical composition comprising: about 120 mg/mL of an anti-CD38 antibody, about 10 mM histidine, about 300 mM sorbitol, about 1 mg/mL methionine, and about 0.04% polysorbate 20, at about pH 5.6.

Embodiment 90 is a pharmaceutical composition comprising: about 120 mg/mL of an anti-CD38 antibody, about 10 mM histidine, about 300 mM D-mannitol, about 1 mg/mL methionine, and about 0.04% Polysorbate 20, at about pH 5.6.

Embodiment 91 is the pharmaceutical composition of any one of embodiments 88-90, wherein the anti-CD38 antibody comprises HCDR1, HCDR2 and HCDR3 amino acid sequences of SEQ ID NOs: 6, 7 and 8, respectively, and LCDR1, LCDR2 and LCDR3 amino acid sequences of SEQ ID NOs: 9, 10 and 11, respectively.

Embodiment 92 is the pharmaceutical composition of any one of embodiments 88-90, wherein the anti-CD38 antibody comprises a heavy chain variable region sequence of SEQ ID NO: 4 and a light chain variable region sequence of SEQ ID NO: 5.

Embodiment 93 is the pharmaceutical composition of any one of embodiments 88-92, wherein the anti-CD38 antibody comprises a heavy chain sequence of SEQ ID NO: 12 and a light chain sequence of SEQ ID NO: 13.

Embodiment 94 is the pharmaceutical composition of any one of embodiments 88-93, wherein the anti-CD38 antibody is of an IgG1 isotype.

Embodiment 95 is a method of providing subcutaneous administration of an anti-CD38 antibody to a subject in need thereof, the method comprising subcutaneously administering to the subject a pharmaceutical composition comprising the anti-CD38 antibody and a pharmaceutically acceptable carrier, wherein the anti-CD38 antibody is subcutaneously administered without recombinant human hyaluronidase.

The following examples of the invention are to further illustrate the nature of the invention. It should be understood that the following examples do not limit the invention, and the scope of the invention is to be determined by the appended claims.

### EXAMPLES

### Example 1: Clinical Study to Evaluate Safety and Tolerability of Subcutaneously Administered Daratumumab

Daratumumab is a targeted immunotherapy that has been reported to deplete cells that express measurable levels of CD38 by a wide spectrum of mechanisms, including complement dependent cytotoxicity (CDC), antibody dependent cell mediated cytotoxicity (ADCC), and antibody dependent cellular phagocytosis (ADCP).

To evaluate the pharmacodynamic (PD) effect of JNJ-54767414 (daratumumab) in healthy normal volunteers, a randomized, double-blind, placebo-controlled single ascending dose study in healthy male and female participants aged 18 to 55 years old was conducted. Fifty-four participants were enrolled in the study. Participants were randomized to receive a single treatment of either daratumumab or placebo. Nine participants were included in each dosage group (ranging between 10 mg to 400 mg), with six participants receiving daratumumab and three participants receiving placebo.

The change in CD38 expression levels and the duration of depletion of CD38 expressing peripheral white blood cells (e.g., plasmablasts/plasma cells (PB_PC), natural killer (NK) cells, lymphocytes, etc.) were measured over time. Immune cell profiling by flow cytometry was performed on whole blood samples following a single subcutaneous administration of daratumumab on Days 1, 2, 4, 8, 15, 22, 29, and every two weeks thereafter until Day 225 post-dose.

Study treatment was initiated with H1 receptor antihistamine and acetaminophen premedication, but without corticosteroid premedication. In particular, oral antipyretics (650 mg to 1000 mg of acetaminophen) and oral or intravenous antihistamine (25 mg to 50 mg of diphenhydramine or equivalent) were administered to each participant. Daratumumab (or placebo) was administered about 1 hour after the premedication by subcutaneous administration. In particular, study participants were subcutaneously administered a total dosage of 0 mg (placebo), 10 mg, 25 mg, 50 mg, 100 mg, 200 mg, or 400 mg of daratumumab formulated in a composition according to an embodiment of the invention. The total dosage was administered in up to four (4) subcutaneous injections in the peri-umbilical area, or alternatively in the thigh. Daratumumab was administered without recombinant human hyaluronidase (rHuPH20).

Depletion of CD38⁺ PB_PC cells, NK cells, total lymphocytes, T lymphocytes, B lymphocytes and monocytes was monitored. The extent to which JNJ-54767414 (daratumumab) depleted or reduced the number and percentage of NK cells (as defined by CD56 positivity) and/or PB_PC cells provided information for the stopping criteria. In short, the median depletion of NK cells and/or PB_PC cells were not to exceed 80% of baseline for longer than 4 weeks.

The safety and tolerability of daratumumab were monitored by physical examinations, local injection site reactions, injection-related reactions/allergic reactions, electrocardiograms, clinical laboratory tests, vital signs, and adverse events. Safety was monitored through day 141 of the study, or for an extended follow-up period if required, based on the assessment of any NK cell and/or plasma cell depletion and/or observation of any other adverse event.

Fresh, whole blood samples were collected from the participants for pharmacokinetic (PK) and pharmacodynamic (PD) assessments, immunogenicity studies, and evaluation of PB_PC and NK cell depletion, as described in the examples below. Flow cytometric analysis were performed on Days 1, 2, 4, 8, 15, 22, 29, and then every 2 weeks through Day 225.

### Example 2: Pharmacodynamic Effects of Subcutaneous Daratumumab Administration

The effects of daratumumab on CD38 expression levels and CD38 expressing cell counts (e.g., percent total of NK cells, PB_PC cells, total lymphocytes, B-cells and T-cells) were evaluated in blood samples collected from participants administered daratumumab as described in Example 1. Baseline CD38 expression profiles were established. The depletion of CD38⁺ NK cells and plasma cells was monitored by flow cytometry.

TABLE 1
CD38 Expression Levels in the Various Cell Populations at Baseline

| | Total Cell Counts | CD38⁺Cell Counts | % CD38 Cells |
|---|---|---|---|
| | Median | Median | Median |
| Cell Types | (low; high) | (low; high) | (low; high) |
| Parent PB_PCs | 30,068 | 21,315 | 73 |
| | (1,862; 117,271) | (1,123; 107,532) | (49; 97) |
| Actual PB_PCs | 738 | 738 | 100 |
| (CD38bright) | (22; 13,100) | (22; 13,100) | (100; 100) |
| NK Cells | 117,855 | 108,438 | 95 |
| | (23,867; 551,537) | (23,450; 545,099) | (71; 99) |
| Lymphocytes | 1,343,542 | 816,463 | 61 |
| | (795,537; 3,119,674) | (454,722; 2,023,120) | (40; 90) |
| T Cells | 866,408 | 457,606 | 53 |
| | (428,024; 2,513,103) | (204,886; 1,542,249) | (30; 88) |
| B Cells | 170,197 | 149,151 | 88 |
| | (71,780; 543,709) | (60,322; 451,434) | (57; 98) |
| Monocytes | 18,751 | 11,486 | 71 |
| | (2,987; 71,074) | (2,433; 44,415) | (34; 99) |

PD Effect on NK CD56⁺ Cells (CD45⁺CD3⁻CD56⁺CD38⁺ cells)

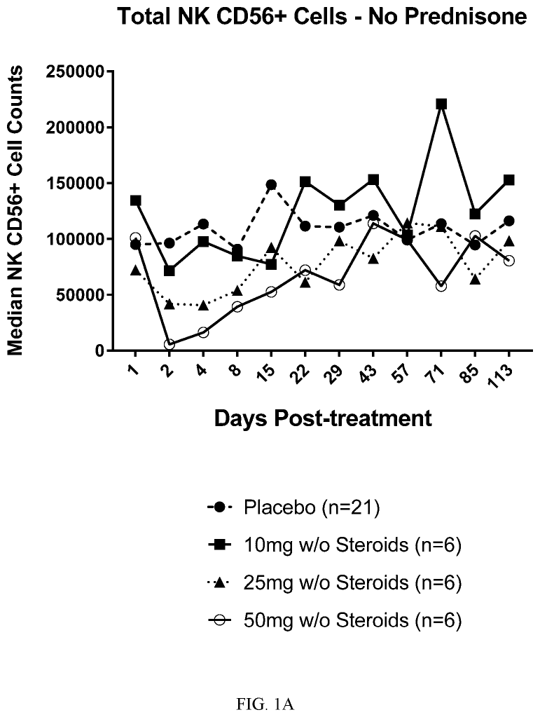

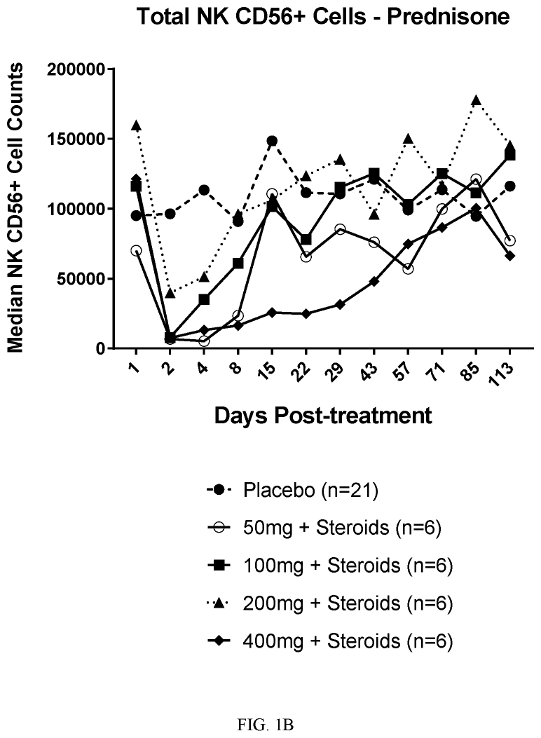

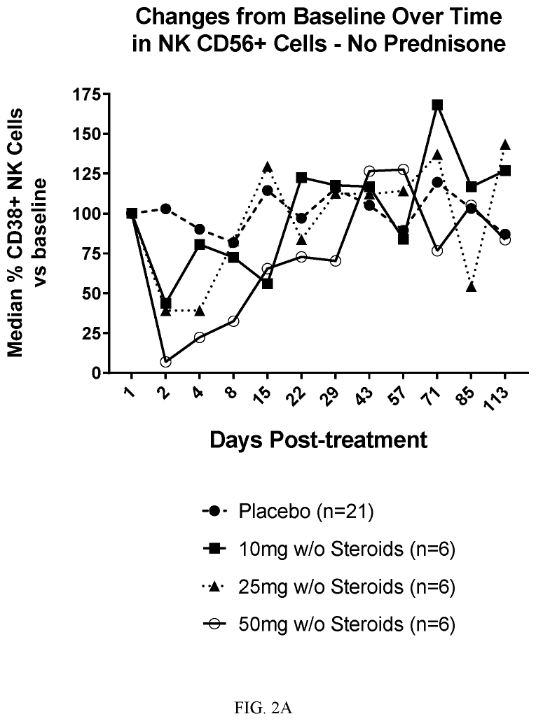

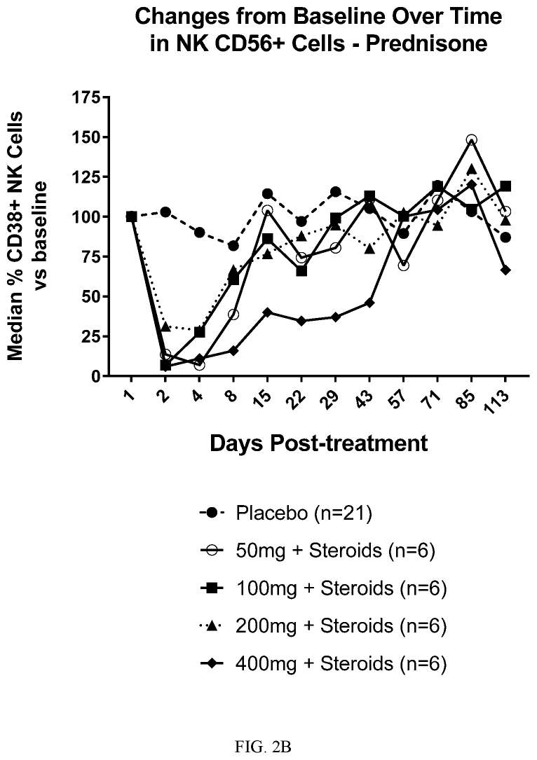

CD38 was expressed on approximately 95% of CD56+NK cells (Table 1). In subsequent analyses, NK cells with phenotype CD45+CD3−CD56+CD38+ were assessed. FIGS. 1A-1B illustrate that very rapidly, within one day after administration of ≥10 mg of daratumumab, the cell counts of CD38⁺CD56⁺ NK cells began to decrease, which equated to a >90% change from baseline on Day 2 post-treatment compared to placebo (FIGS. 2A-2B). In measuring the effect of daratumumab on percentage of CD38⁺CD56⁺ NK cells, the greatest change from baseline was observed on Day 7, where several doses of daratumumab caused no more than an approximately 20% decrease in CD38⁺CD56⁺ NK cell percentages. The observation that changes in the number of CD38⁺CD56⁺ NK cells did not mirror changes in the percentages of the same CD38 expressing CD56⁺ NK cells is likely due to the very high level of CD38 expression in this cell population. Recovery of CD38⁺CD56⁺ NK cells toward baseline levels began relatively quickly (within 7-14 days) across dose groups, although CD38⁺CD56⁺ NK cell recovery in the 400 mg dose group (FIGS. 1A-1B and 2A-2B) was moderately slower compared to other doses. In general, however, the return to baseline of CD38⁺CD56⁺ NK cell levels was generally achieved within 21 days post-dose.

PD Effects on PB_PC Cells (CD45⁺CD3⁻CD19⁺CD20⁻CD27⁺IgD⁻CD38⁺ cells)

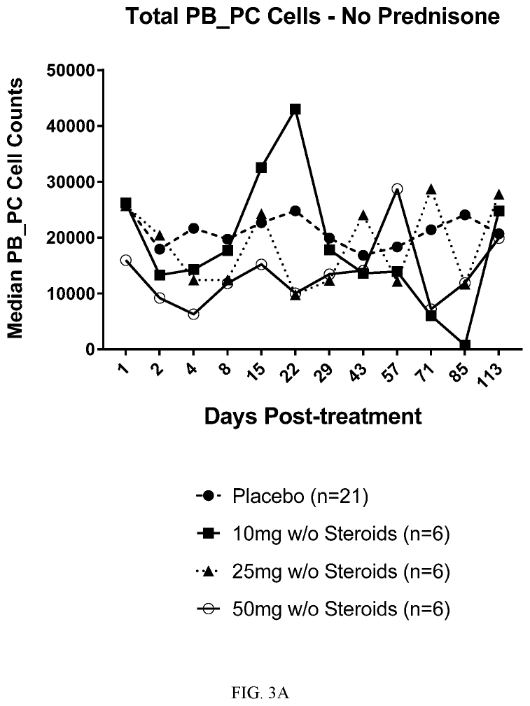

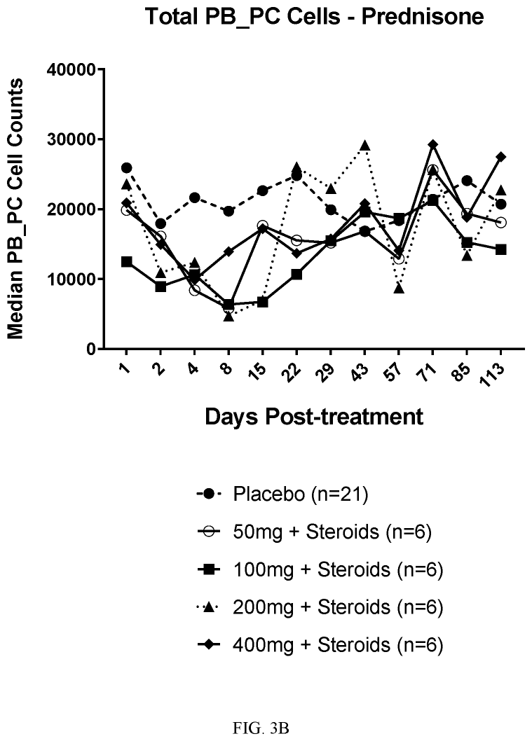

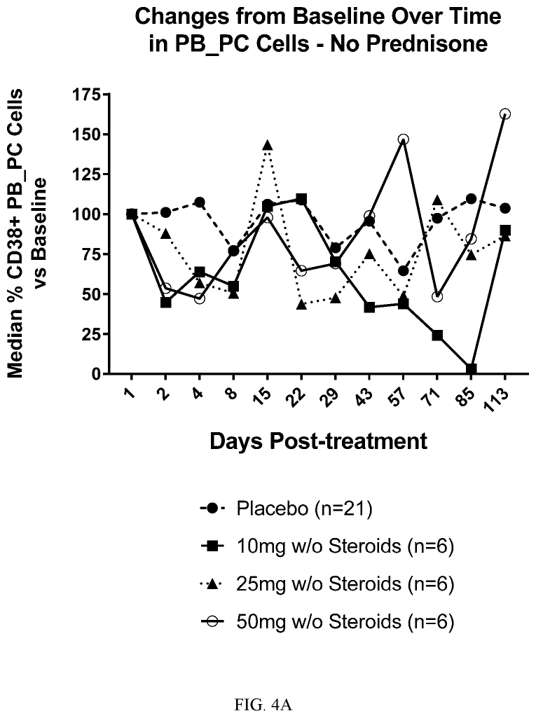

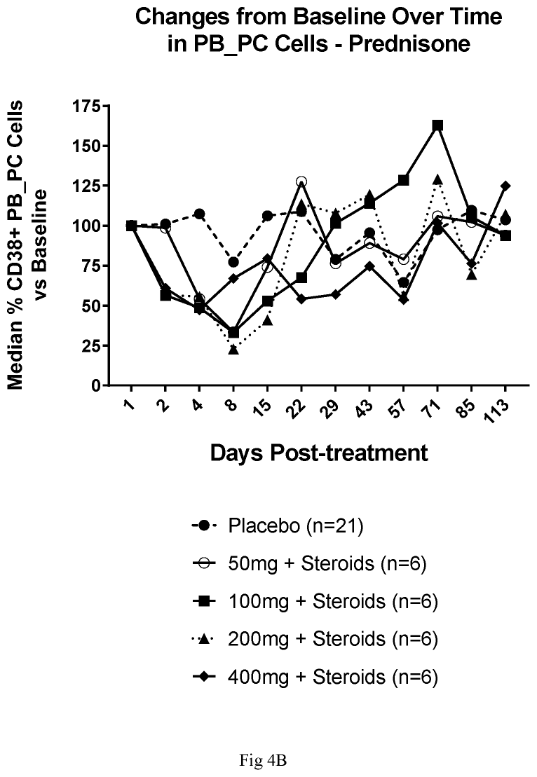

While the percentage of NK cells in the peripheral blood was between 2-5%, the percentage of PB_PC cells was much smaller, at 0.1-0.5% of peripheral blood leukocytes. PB-PC cells were defined as CD45⁺CD3⁻CD19⁺CD20⁻CD27⁺IgD⁻CD38⁺ in this study. At baseline, CD38 was expressed at a median percentage of 70-88% in the PB_PC population across subjects participating in this study (Table 1). Similar to NK cells, the cell counts and percentage of CD38⁺ PB_PC cells started to diminish within 2 days, but maximal depletion was observed on Day 8 post-treatment with doses of daratumumab≥25 mg (FIGS. 3A-3B). The maximal depletion in CD38⁺ PB_PC cell counts was observed on Day 8 in the 200 mg dose group with an 80% reduction in actual cell counts and a median drop of 85% in percentage of CD38⁺ PB_PC cells (FIGS. 4A-4B). On Day 8, the change from baseline in CD38+ PB_PCs cell counts and percentages was 80%. Recovery of cell numbers and percentages occurred relatively quickly, within 14-21 days, for all daratumumab dose groups tested.

Other white blood cell populations were also monitored in this study, including total lymphocytes, T cells, B cells, and monocytes.

PD Effects on Total Lymphocytes (CD45⁺CD38⁺ cells)

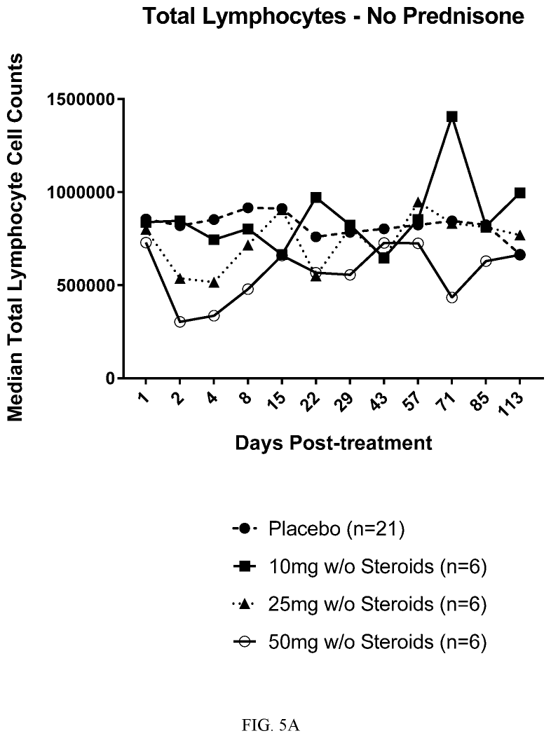

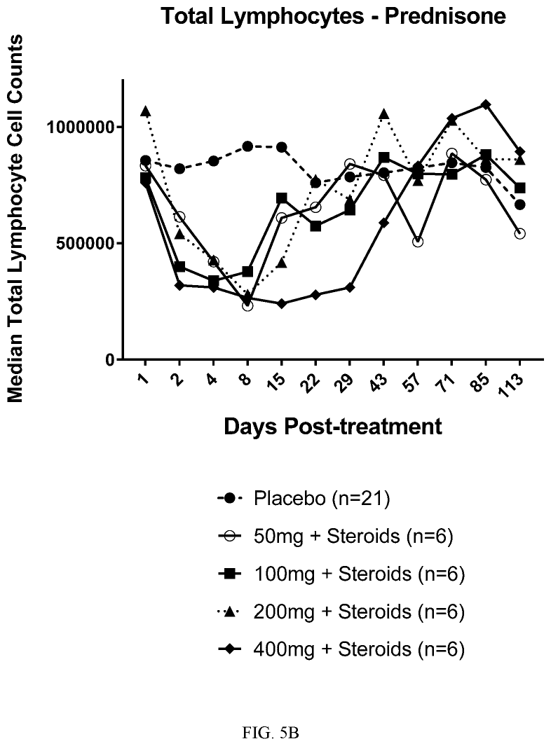

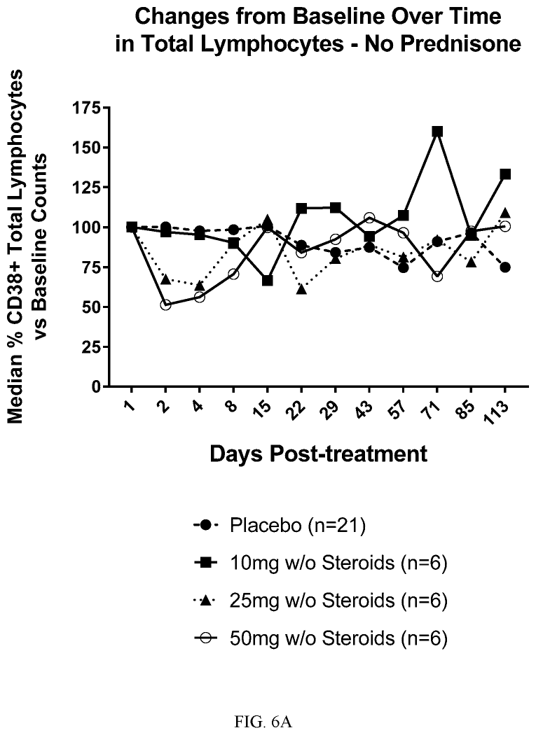

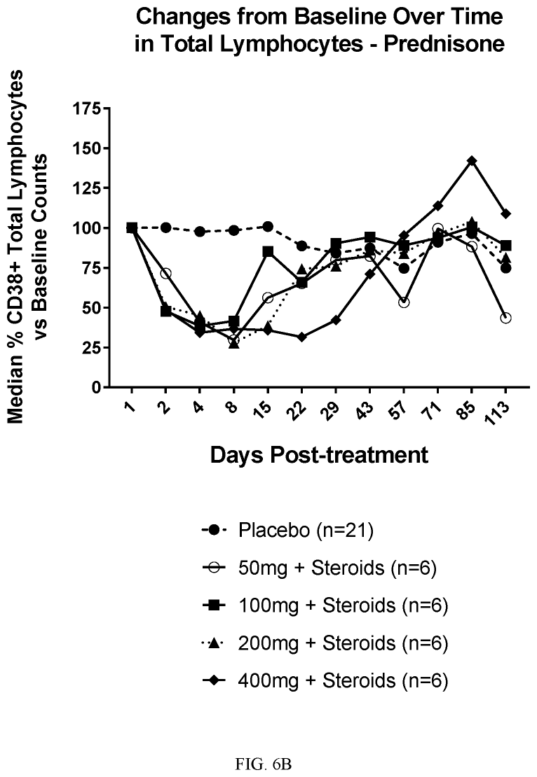

CD38 was present on 50-70% of total CD45⁺ lymphocytes (Table 1). Within 1-2 days, the total number and percentage of CD38⁺ CD45⁺ lymphocytes started to decrease after administration of daratumumab (at doses≥25 mg) (FIGS. 5A-5B). The maximal depletion of CD38⁺ CD45⁺ lymphocytes occurred on Day 8 (FIGS. 5A-5B and 6A-6B). At doses≥50 mg, the cell counts were reduced by approximately 70% from baseline, and the percentages of CD38⁺CD45⁺ lymphocytes were reduced from baseline by approximately 80% (FIG. 6B). Recovery of CD38⁺CD45⁺ lymphocyte cell numbers and percentages occurred relatively quickly, within 14-21 days, for daratumumab dose groups≤200 mg. However, the recovery of lymphocytes after administration of 400 mg daratumumab was slower, and CD38⁺ lymphocyte cell counts and percentages did not approach baseline levels again until Day 43 (FIGS. 5B and 6B).

PD Effects on T Lymphocytes (CD45⁺CD3+CD38⁺ cells)

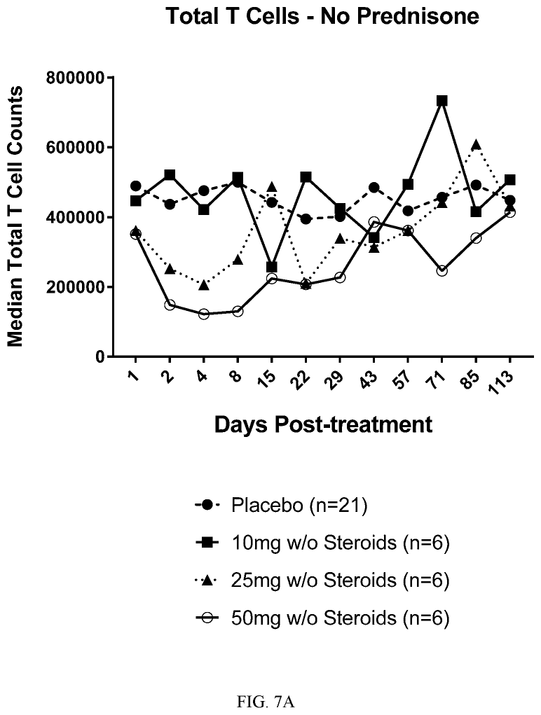

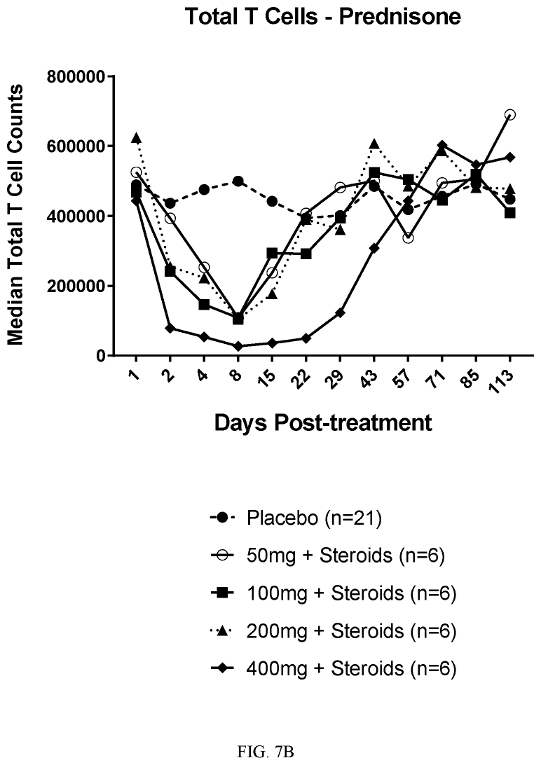

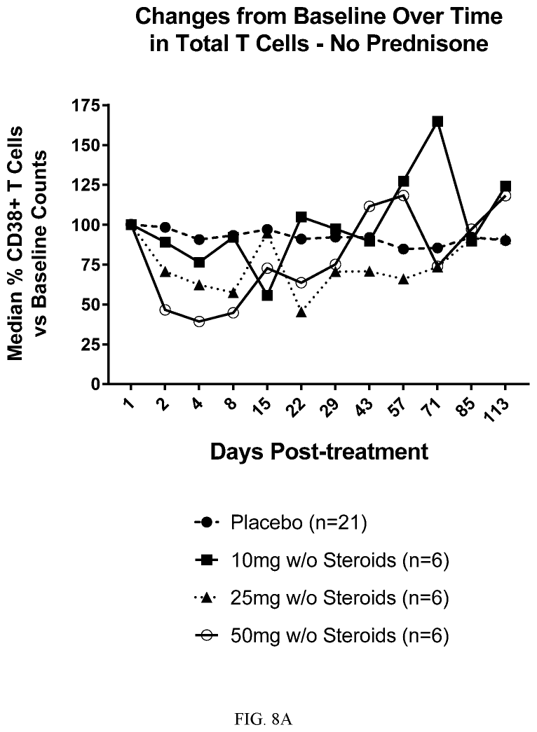

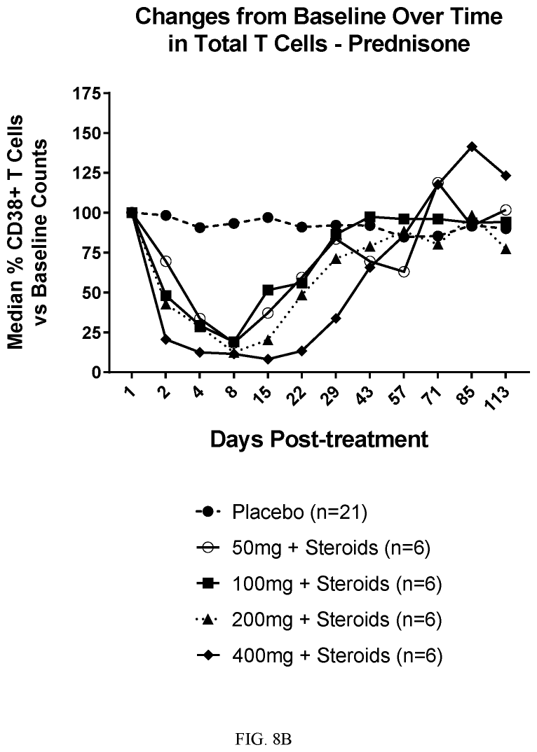

At baseline, the percentage of CD38⁺ expression in total T lymphocytes as defined by CD56⁻CD14⁻CD19⁻CD45⁺CD3⁺ was in the range of 40-60% (Table 1). Within 1-2 days after dosing with daratumumab, there was an observable reduction in the cell numbers and percentages of CD38⁺ T cells (FIGS. 7A-7B). The maximal reduction in total T lymphocyte counts and percentages by daratumumab administration occurred on Day 8, with the 400 mg dose showing the greatest reduction from baseline (90% reduction of CD38⁺ T cells; FIGS. 7B and 8B). Recovery of total T cells started to occur 14 days post-dose, except in the 400 mg daratumumab dose group, where return to baseline T cell counts and percentage was slower (FIGS. 7A-7B and 8A-8B). While CD38⁺ T cells started to recover on Day 22 after administration of the 400 mg dose, return to baseline level cell counts and percentages occurred >Day 43 (FIGS. 7B and 8B).

PD Effects on B Lymphocytes (CD45⁺CD19⁺CD38⁺ cells)

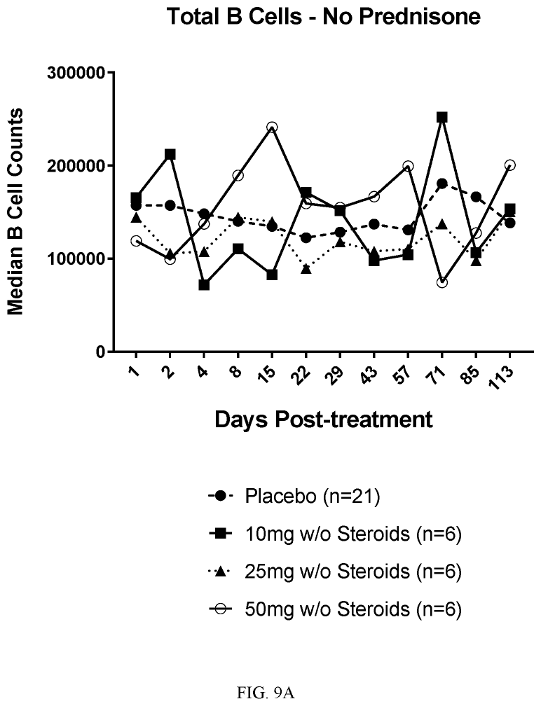

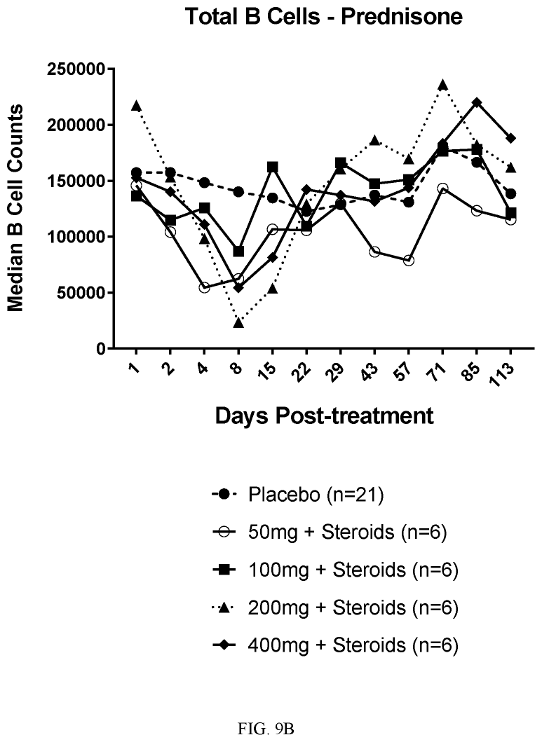

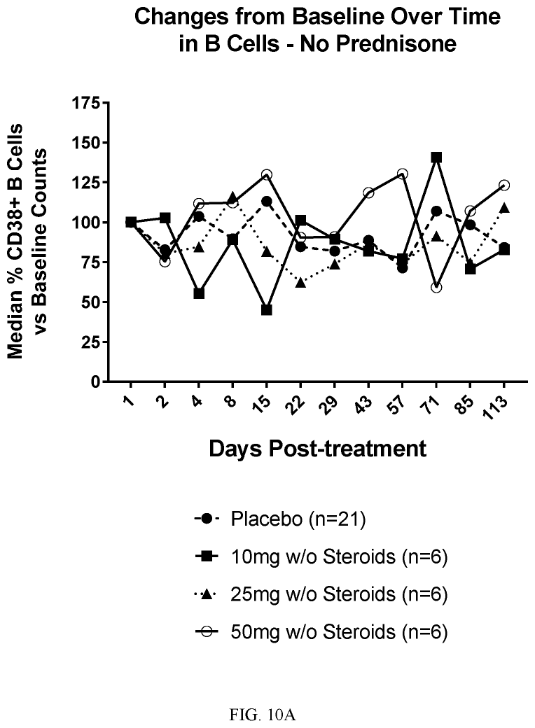

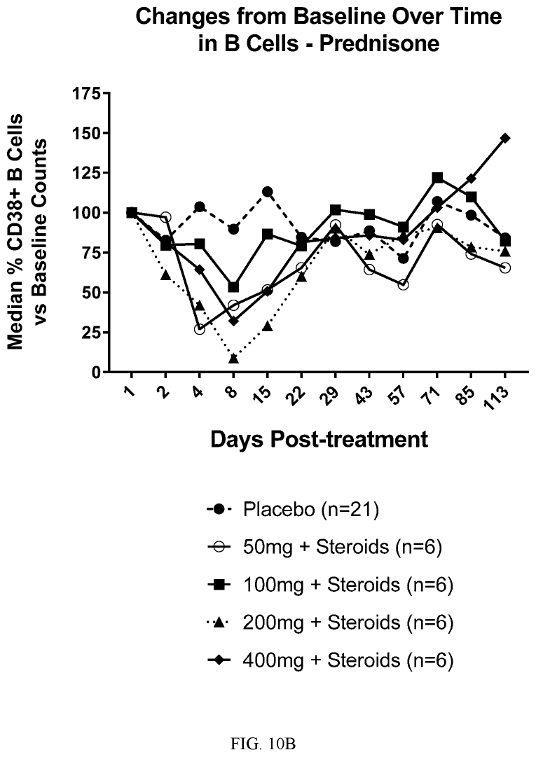

In CD19⁺ B cell populations, the baseline expression level of CD38 measured in the subjects enrolled in this study ranged from 82-95% (Table 1). Daratumumab reduced the cell numbers and percentages of CD38⁺CD19⁺ cells at doses≥50 mg within 2-4 days (FIGS. 9A-9B); however, maximal depletion of CD38⁺CD19⁺ B cells occurred on Day 8 where a 90% decrease from baseline cell counts and percentages resulted from a 200 mg dose administration (FIGS. 9B and 10B). Interestingly in this B cell population, the 400 mg dose of daratumumab did not reduce cell numbers or percentages (˜70%) more than the 200 mg dose (FIGS. 9B and 10B). Unlike other cell populations, recovery of CD38⁺CD19⁺ B cells occurred within a relatively short timeframe as baseline levels were achieved in these subjects within 21 days across all daratumumab doses.

PD Effects on Monocytes (CD45⁺CD3⁻CD19⁻CD56⁻CD14⁺CD16⁻CD38⁺ cells)

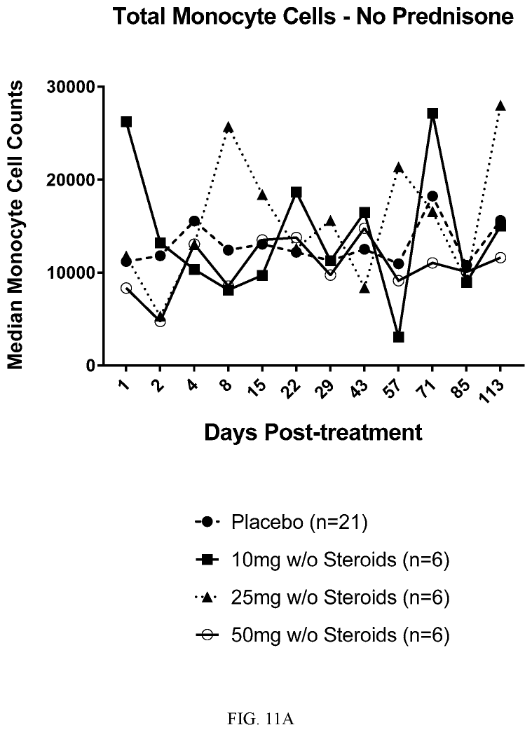

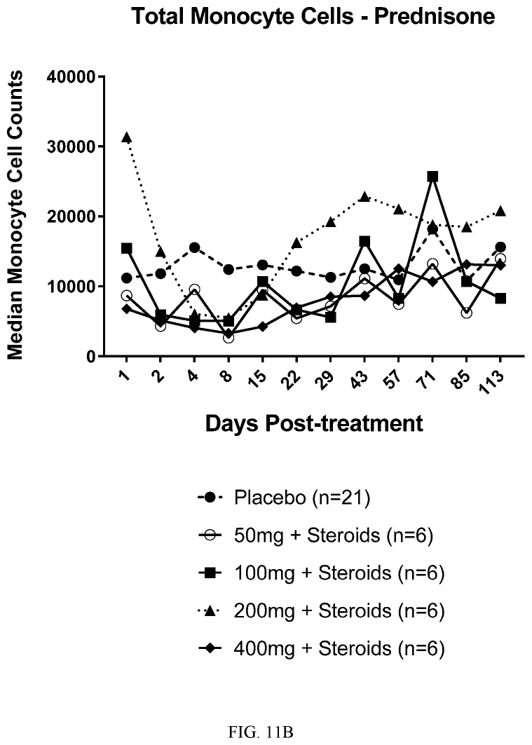

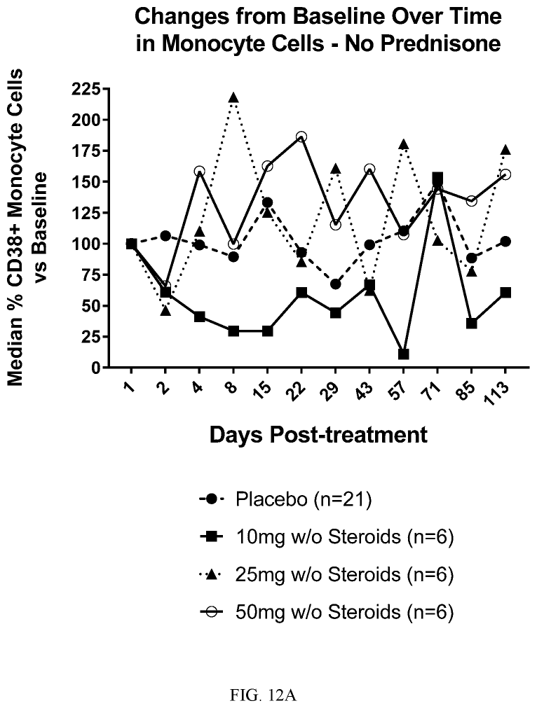

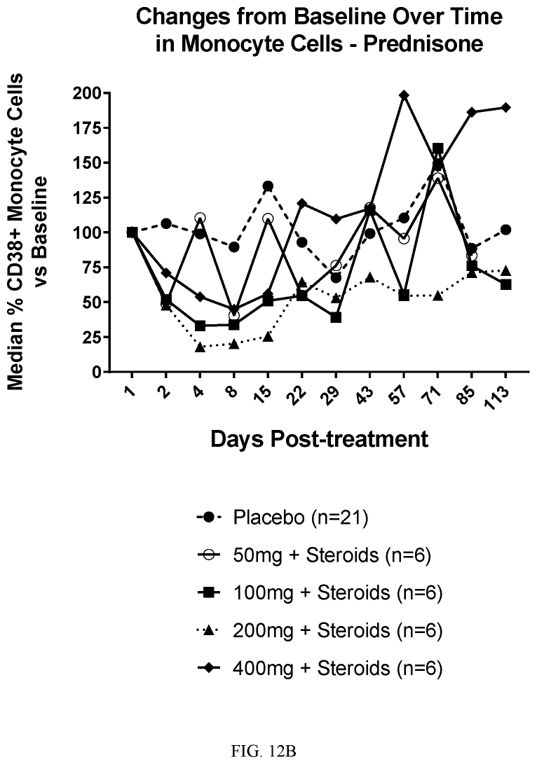

The effect of increasing doses of daratumumab on monocytes was also monitored (FIGS. 11A-11B and 12A-12B). CD38 expression on CD45⁺CD3⁻CD19⁻CD56⁻CD14⁺CD16⁻monocytes in subjects enrolled in this study ranged from 50-80% at baseline (Table 1). While blood monocytes were present in small numbers (2-8%), daratumumab reduced the cell counts and percentages of CD38⁺ monocytes within 2-7 days (FIGS. 11A-11B). The numbers and percentages of CD38⁺CD45⁺CD3⁻CD19⁻CD56⁻CD14⁺CD16⁻ monocytes decreased to a maximal level by day 7 after administration of daratumumab, and return of monocytes to baseline levels usually occurred within 21 days. In the monocyte population, the magnitude of reduction induced by low and high doses of daratumumab overlapped and were too varied to draw definitive conclusions about the effect of daratumumab on these cells.

### Example 3: Clinical Study to Evaluate Effect of Premedication with Corticosteroid on Safety and Tolerability of Subcutaneously Administered Daratumumab

The effect of premedication with a corticosteroid on the safety and tolerability of subcutaneously administered daratumumab was evaluated. Study participants were administered prednisone (40 mg, orally administered) 1 to 2 hours prior to subcutaneous administration of daratumumab. Daratumumab was subcutaneously administered as described in Example 1. Corticosteroid dosages were modified as needed to prevent systemic injection related reactions.

Corticosteroids were administered to subjects who received doses of daratumumab≥50 mg in order to manage injection-related reactions. More specifically, subjects in the 50-200 mg dose groups received 40 mg of corticosteroids prior to drug delivery; whereas the subjects in the 400 mg dose group received 40 mg of corticosteroids prior to drug delivery and 20 mg of corticosteroids directly afterwards. To determine whether corticosteroids themselves might impact the percentages of CD38⁺ cells across different populations, data from N=9 placebo subjects that did not receive corticosteroids were combined for comparison to data from N=12 placebo subjects that received equivalent corticosteroid dosing to subjects receiving daratumumab. As shown in FIGS. 1A-1B, 2A-2B, 3A-3B, 4A-4B, 5A-5B, 6A-6B, 7A-7B, 8A-8B, 9A-9B, 10A-10B, 11A-11B, and 12A-12B, the percentages of CD38⁺ NK cells, PB_PC cells, total lymphocytes, T cells, B cells, and monocytes were comparable in subjects that received corticosteroids versus those that did not receive corticosteroids, suggesting that addition of corticosteroids to subjects did not significantly alter the percentages of peripheral blood cells being monitored in this study for daratumumab PD effects.

Results from Examples 1-3 demonstrated that the PD effects of daratumumab can be measured by flow cytometric analysis of CD38 expression across multiple cell types in the peripheral blood. Increasing doses of daratumumab were shown to decrease the number and percentages of CD38⁺ PB_PC cells, NK cells, total lymphocytes, T cells, B cells, and monocytes within 7 days post-administration. Across cell populations, daratumumab started to reduce CD38⁺ cell counts and percentages relatively quickly (within 1-2 days) with maximal depletion usually observed by 7 days after a single dose of daratumumab≥25 mg. In CD38⁺ cell populations, recovery toward baseline levels usually began within 14-21 days after administration of daratumumab at doses<200 mg. Compared to lower dose groups, recovery of CD38⁺ cell counts and percentages to baseline levels was generally slower in the 400 mg daratumumab treated subjects. Overall, results from this study demonstrate that in healthy normal volunteers, daratumumab broadly depletes CD38⁺ cell populations in the peripheral blood but to a different extent across cell subtypes.

### Example 4: Immunogenicity Evaluation

Anti-daratumumab antibodies are evaluated in serum samples collected from participants administered daratumumab according to the study described in Examples 1 and 3. In particular, neutralizing antibodies (NAbs) against daratumumab in human serum samples derived from daratumumab dosed subjects, who test positive for anti-daratumumab antibodies, are determined.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method of providing subcutaneous administration of an anti-CD38 antibody to a subject in need thereof, the method comprising subcutaneously administering to the subject a pharmaceutical composition comprising the anti-CD38 antibody and a pharmaceutically acceptable carrier, wherein the total dosage of the anti-CD38 antibody is about 10 mg to about 2,400 mg per administration.

2. The method of claim 1, wherein the total dosage of the anti-CD38 antibody is about 10 mg to about 400 mg per administration.

3. The method of claim 1, wherein the total dosage of the anti-CD38 antibody is administered in a single subcutaneous injection.

4. The method of claim 1, wherein the total dosage of the anti-CD38 antibody is administered in two to five subcutaneous injections.

5. The method of claim 1, wherein a corticosteroid is administered to the subject prior to the administration of the anti-CD38 antibody, and is optionally re-administered subsequent to the administration of the anti-CD38 antibody.

6. The method of claim 5, wherein the corticosteroid is administered orally.

7. The method of claim 5, wherein the corticosteroid is prednisone.

8. The method of claim 1, wherein the administration of the anti-CD38 antibody results in less than 80% depletion of natural killer (NK) cells or plasma cells four (4) weeks after administration of the anti-CD38 antibody.

9. The method of claim 1, wherein the anti-CD38 antibody is subcutaneously administered without recombinant human hyaluronidase.

10. The method of claim 1, wherein the anti-CD38 antibody is subcutaneously administered with recombinant human hyaluronidase.

11. The method of claim 10, wherein the hyaluronidase is rHuPH20 (SEQ ID NO: 22).

12. A method of providing treatment of an autoimmune disease in a subject in need thereof, the method comprising subcutaneously administering to the subject a pharmaceutical composition comprising an anti-CD38 antibody and a pharmaceutically acceptable carrier, wherein the total dosage of the anti-CD38 antibody is about 10 mg to about 2,400 mg per administration.

13. The method of claim 12, wherein the total dosage of the anti-CD38 antibody is about 10 mg to about 400 mg per administration.

14. The method of claim 12, wherein the total dosage of the anti-CD38 antibody is administered in a single subcutaneous injection.

15. The method of claim 12, wherein the total dosage of the anti-CD38 antibody is administered in two to five subcutaneous injections.

16. The method of claim 12, wherein a corticosteroid is administered to the subject prior to administration of the anti-CD38 antibody, and is optionally re-administered subsequent to administration of the anti-CD38 antibody.

17. The method of claim 16, wherein the corticosteroid is administered orally.

18. The method of claim 16, wherein the corticosteroid is prednisone.

19. The method of claim 12, wherein the administration of the anti-CD38 antibody results in less than 80% depletion of natural killer (NK) cells or plasma cells four (4) weeks after administration of the anti-CD38 antibody.

20. The method of claim 12, wherein the anti-CD38 antibody is subcutaneously administered without recombinant human hyaluronidase.

21. The method of claim 12, wherein the anti-CD38 antibody is subcutaneously administered with recombinant human hyaluronidase.

22. The method of claim 21, wherein the hyaluronidase is rHuPH20 (SEQ ID NO: 22).

23. The method of claim 12, wherein the autoimmune disease is selected from the group consisting of arthritis, rheumatoid arthritis (RA), psoriatic arthritis, ankylosing spondylitis, ulcerative colitis, plaque psoriasis, systemic lupus erythematosus (SLE), lupus nephritis, antineutrophil cytoplasmic antibody (ANCA) associated vasculitis, myasthenia gravis, progressive multiple sclerosis, IgG4 related diseases, Sjogren's syndrome, immune thrombocytopenic purpura, transplant rejection, inflammatory bowel disease and Crohn's disease.

24. The method of claim 23, wherein the autoimmune disease comprises or consists of RA.

25. The method of claim 23, wherein the autoimmune disease comprises or consists of SLE.

26. The method of claim 1, wherein the anti-CD38 antibody comprises heavy chain complementarity determining region 1 (HCDR1), HCDR2 and HCDR3 amino acid sequences of SEQ ID NOs: 6, 7 and 8, respectively, and light chain complementarity determining region 1 (LCDR1), LCDR2 and LCDR3 amino acid sequences of SEQ ID NOs: 9, 10 and 11, respectively.

27. The method of claim 1, wherein the anti-CD38 antibody comprises a heavy chain variable region sequence of SEQ ID NO: 4 and a light chain variable region sequence of SEQ ID NO: 5.

28. The method of claim 1, wherein the anti-CD38 antibody comprises a heavy chain sequence of SEQ ID NO: 12 and a light chain sequence of SEQ ID NO: 13.

29. The method of claim 1, wherein the anti-CD38 antibody is of an IgG1 isotype.

30. The method of claim 1, wherein the subject is a human.
